(19) United States
(12) Patent Application Publication
(10) Pub. No.: US 2026/0366065 A1
(43) Pub. Date: Nov. 19, 2026

(54) COMPOSITIONS, SYSTEMS AND METHODS FOR MULTIPLEX DETECTION OF TARGET BIOMARKERS IN A SAMPLE

(71) Applicant: KING ABDULLAH UNIVERSITY OF SCIENCE AND TECHNOLOGY, Thuwal (SA)

(72) Inventors: Dana Zaid ALSULAIMAN, Thuwal (SA); Erol HASAN, Thuwal (SA); Duruo MAO, Thuwal (SA)

(21) Appl. No.: 19/477,783

(22) PCT Filed: Apr. 24, 2024
(86) PCT No.: PCT/IB2024/054024

§ 371 Date: Oct. 23, 2025

Related U.S. Application Data
(60) Provisional application No. 63/497,957, filed on Apr. 24, 2023.

(51) Int. Cl. C12Q 1/6825; C12Q 1/6876

(52) U.S. Cl. CPC C12Q 1/6825; C12Q 1/6876; C12Q 2600/16; C12Q 2600/178

(57) ABSTRACT

Compositions, systems and methods for multiplex detection of target biomarkers in a simple are provide, offering highly sensitive and multiplexed detection. The compositions and methods are preferably isothermal, label-free, and enzyme-free. The compositions include (i) a DNA carrier molecule comprising (decorated along its length—in a sequence-specific manner) with sequences complementary to sequences in the PNA based anchor and (ii) bifunctional PNA-based anchors which are complementary on the one end to the sequences the analytes of interest and complementary on the other end to sequences on the DNA carrier, the complementarity being provided by a first and a second PNA molecule. The disclosed systems and method include a nanopore sensor.

### CROSS REFERENCE TO RELATED APPLICATIONS

The present application claims priority to U.S. Application No. 63/497,957, filed Apr. 24, 2023, the disclosures of which is incorporated herein by reference in its entirety.

### FIELD OF THE INVENTION

This invention is generally in the field of compositions and methods for multiplex detection of analytes.

### BACKGROUND OF THE INVENTION

The ability to do high-throughput, sensitive and multiplex detection of low abundance biomarkers in biological, environmental, and other fluid samples can transform the way many diseases and pathogens are detected, diagnosed, and monitored, including cancer. Gold-standard techniques for cancer diagnosis still rely upon taking a tissue biopsy of the primary tumor site—a highly invasive procedure which can cause health complications and cannot be performed repeatedly over time. Therefore, detecting cancer biomarkers from ‘liquid biopsies’, which rely upon minute volumes of biofluid samples, offers promising potential to revolutionize cancer detection while providing real-time analysis and dynamic monitoring of disease progression (Kawaguchi et al., Int. J. Mol. Sci., 17 (2016); Siravegna, et al., Nat. Rev. Clin. Oncol. 14:531-548 (2017); Ono et al., J. Clin. Med. Res.4:1890-1907 (2015)).

Among the clinical biomarkers that have gained interest, microRNAs (miRNAs) have emerged as promising non-invasive biomarkers for cancer (Ambros, V., Nature 431:350-355 (2004); Bartel, Cell 116:281-297 (2007)). They are a class of short non-coding RNA (19-25 nucleotides in length), and show potential as diagnostic and prognostic biomarkers due to their gene regulatory functions and dysregulated patterns in many diseases including cancer (Calin, Nat. Rev. Cancer 2006, 6, 857-866). Moreover, their expression profiles are tissue, developmental and disease specific (Lu, et al., Nature 2005, 435, 834-838; Iorio, M. Cancer Res. 2005, 65, 7065-7070). In addition to providing valuable clinicopathological information, miRNAs possess innate properties which make them attractive biomarkers e.g., they are present in all human body fluids and remain well-preserved in clinical specimens including blood plasma, serum, and urine (Pritchard, et al., Nat. Rev. Genet. 2012, 13, 358-369; Weber, et al., Clin. Chem. 2010, 56, 1733-1741). Absolute quantification of miRNA remains a significant challenge due to their low circulating concentrations (nanomolar to picomolar regime), short lengths, high sequence homology among family members, and variable expression levels in biological samples.

The gold-standard strategies for miRNA detection, including reverse-transcription (RT) qPCR and microarrays (Liu, et al., Nat. Protoc. 2008, 3, 563-578), involve complex multistep procedures, high turnaround times, and the use of costly instrumentation as well as trained personnel. While RT qPCR is a reliable and sensitive technique, it requires enzymatic amplification, expensive dual-labeled probes (e.g. Taqman probe), and thermal cycling. It also offers limited multiplexing capability and is particularly costly if more than one miRNA must be assayed. The expression analysis of miRNA with hybridization-based microarrays enables much greater multiplexing but requires pre-labelling of the target through dephosphorylating its 5′ end followed by ligation of a fluorescently tagged oligonucleotide or short oligonucleotide strand using T4 ligase enzyme (Graybill, R. et al., Anal. Chem. 2016, 88, 431-450). This labeling procedure is highly complex, costly, and typically prone to bias (de Planell-Saguer, et al., Clin. Biochem. 2013, 46, 869-878). Recently, Nanostring™ has also developed an assay for miRNA expression profiling called nCounter® that does not require RT or amplification, but it still involves multiple complex steps (requiring enzymes and data processing) which are both time-consuming (~2-day protocol) and expensive (Kulkarni, M., Curr. Protoc. Mol. Biol. 2011, Chapter 25, Unit25B.10).

High turnaround time, non-isothermal conditions as well as the requirement for enzymes, fluorescent labels and bulky expensive equipment severely limits the use of these molecular techniques in clinical and other applications. There remains an urgent and unmet need (clinical and otherwise) to develop biomarker detection tools which not only offer high sensitivity and robustness, but also enable multiplex detection of target biomarkers in a simple, cost-effective, and timely manner. Such tools will radically transform the way diseases e.g., cancer, and pathogens are diagnosed, detected, and monitored through the use of non-invasive and highly sensitive ‘liquid biopsy’ tests.

Therefore, compositions for multiplex detection of target biomarkers in a simple are provided.

Also provided are methods for multiplex detection of target biomarkers in a sample.

### SUMMARY OF THE INVENTION

Compositions, systems, and methods for multiplex detection of target analytes such as biomarkers in a sample are provided, offering highly sensitive and multiplexed detection.

The compositions and methods are preferably isothermal, label-free, and enzyme-free. The compositions include (i) a DNA carrier molecule including (decorated along its length—in a sequence-specific manner) sequences complementary to sequences in a Xeno-nucleic acid (XNA) based anchor and (ii) a bifunctional XNA-based probe. The bifunctional XNA-based probe includes two functional domains preferably, separated by a linker: (a) a XNA sequence which is complementary to a sequence on the DNA carrier (herein, XNA-based anchor”); and (b) a binding partner for an analyte e.g., a bioreceptor specific to the analyte of interest (herein, a “hook”).

In some forms, the XNA-based anchor is a peptide nucleic acid (PNA). However, the XNA-based anchor could be selected from the group consisting of HNA (1,5-Anhydrohexitol nucleic acid), CeNA (Cyclohexene nucleic acid), TNA (Threose nucleic acid), GNA (Glycol nucleic acid), LNA (Locked nucleic acid) or FANA (Fluoroarabino nucleic acid).

In some forms, the hook is a nucleic acids such as XNA, for example, a PNA. However, the hook can include a peptide, protein, polysaccharide, which is a binding partner for an analyte of interest.

In some forms, the linker is a flexible linker, and is preferably selected to be stable under the reaction conditions for the say e.g., a short linker. In one embodiment, the linker is a polymeric linker e.g., polyethylene glycol. In some forms, the linker is a peptide linker such as a short, flexible peptide linker.

In some forms, the XNA-based anchor and the hook are both PNAs. For example, in one embodiment, the XNA-based probe includes a first and a second PNA molecule separated by a linker.

The XNA is preferably one with the ability to invade a double stranded DNA e.g., an invading PNA. Generally, XNA-based probes are engineered such that one end invades and binds to a dsDNA carrier at sequence-specific pre-defined locations, while the other end (“the hook”) acts as a probe for capture of an analyte of interest. The design, characteristics, and properties of the engineered XNA-based probes allow for the formation of a two-dimensional hybridization complex between the carrier DNA and target analyte e.g., a target biomarker. In some forms, the XNA-based probe includes a third molecule, (such a XNA sequence) functionalized with a suitable moiety e.g., a label (herein, “amplifier XNA”).

The disclosed compositions are designed to be “read out” or “decoded” using single-molecule detection technologies, such as solid-state nanopores for electrical readouts or DNA mapping technologies e.g., nanochannels arrays for optical readouts. Thus, a system for multiplex detection of target biomarkers in a sample is also provided, which includes the disclosed compositions, and a single-molecule detection device e.g., nanopore sensor, preferably, a solid-state nanopore sensor or nanochannel arrays.

The disclosed methods and systems are used in a method for detecting one or more analytes of interest present in a sample, such as a biological sample, including but not limited to blood, plasma, tissue, and other bodily fluids. Optionally, the biological sample is of environmental origin such as from wastewater, soil, or environmental surfaces.

Analytes of interest which can be detected using the disclosed compositions, systems, and methods, include but are not limited to biomarkers such as nucleic acid biomarkers e.g., microRNA, cell-free DNA, mRNA, and ribosomal RNA; proteins e.g., hormones, enzymes, antigens, and antibodies; polysaccharides, viral particles, and other analytes of clinical and environmental interest.

The disclosed method includes contacting a sample with a composition containing a carrier DNA and/or one or more XNA-based probes and optionally, a labelled molecule such as a labelled XNA. For example, the method may involve contacting a raw sample or extracts from the raw sample, such as obtained through an extraction step, with a composition containing a carrier DNA and/or one or more XNA-based probes to isolate the analyte of interest from the raw sample or sample extract.

The disclosed method further includes detecting the positions of the PNA probes in the sample using single-molecule detection devices e.g., nanopore sensors or nanochannel arrays. For example, samples contacted with the disclosed compositions can be “read out” or “decoded” using single-molecule detection technologies, such as solid-state nanopores for electrical readouts or DNA mapping technologies e.g., nanochannels for optical readouts.

### BRIEF DESCRIPTION OF THE DRAWINGS

FIG. 1A-D is a simplified diagram describing the proposed platform, which aims to enable ultrasensitive and multiplex detection of biomarkers from (FIG. 1A) ‘liquid biopsy’ tests using a sensing platform based on a (FIG. 1B) dsDNA carrier decorated with location-encoded fishing probes. The platform can (FIG. 1C) encode clinicopathological information (about analyte levels e.g., biomarker levels) and (FIG. 1D) decode this information in a high-throughput way using a single molecule sensing system e.g., nanopore detection. FIG. 1E is a schematic representing the design of an exemplary fishing probe, including an ‘anchor’ motif which invades the dsDNA carrier at sequence-specific sites and a ‘hook’ motif which can capture or “fish” biomarkers (by acting as a bioreceptor).

FIG. 2A is a schematic representation of an exemplary system as described herein, for location-encoded multiplexed detection of target miRNA using a 2D hybridization complex, and the single molecule sensing method, solid-state nanopore. A carrier dsDNA (10 kbp) is decorated along its length with probes targeting miRNA biomarkers in a location-encoded manner. The probes can detect biomarkers such as miRNA through the formation of a 2D hybridization complex. In the example case shown, miRNA-A, miRNA-B and miRNA-D are upregulated in this cancer type and detected by the system while miRNA-C was down-regulated and thus not detected. This generates a current-time trace or ‘signature of disease’ on the nanopore output, which is characteristic of a cancer patient miRNA profile. The probe consists of a PNA-PNA conjugate, where one PNA acts as the anchor (blue) onto the DNA carrier, while the second acts as the capture (green) detecting the target miRNA (red). To improve the signal-to-noise ratio in the nanopore-based detection system, a bulky amplifier-PNA (purple) is added which is complementary to the other end of the target miRNA. FIG. 2B is a schematic of another exemplary system for the multiplexed detection of analytes e.g., biomarkers using a nanopore readout. A carrier dsDNA (~10 kbp) is decorated along its length with fishing probes targeting biomarkers. Through their ‘hook’ component which can be any bioreceptor e.g. PNA probe or antibody, the probes can capture or “fish” biomarkers. Similar to FIG. 2A, biomarker-A, biomarker-B and biomarker-D are upregulated in this cancer type and detected by the system while biomarker-C was down-regulated and thus not detected, generating a current-time trace on the nanopore output, which is characteristic of a cancer patient biomarker profile (FIG. 2B, right panel). For miRNA detection, a PNA-PNA fishing probe is used such that the ‘hook’ motif PNA (green) is complementary to the target miRNA (red) (see blue circle). For antigen (protein) biomarker detection, a PNA-antibody fishing probe is used where the ‘hook’ motif antibody (blue) can capture the target antigen (purple) (see orange circle).

FIG. 3A is a schematic showing the methodology of solid phase peptide synthesis (SPPS) used for PNA and peptide synthesis. Structure (FIG. 3B) and MALDI-TOF spectrum (FIG. 3C) of an example fluorescently-labelled PNA probe, successfully synthesized in prior work, with sequence (C- to N-terminus): K-K-TGTGACA-fluorescein (MW=2606.59 Da), K=Lysine amino acid (Al Sulaiman, D et al., Angew. Chem. Int. Ed Engl. 2017, 56, 5247-5251).

FIG. 4A is a schematic of an exemplary 2D hybridization complex illustrating nanopore-based validation of anchor-PNA invasion capacity for a single bulky anchor-PNA. FIG. 4B is a schematic of an exemplary system, nanopore-based validation and optimization of location-based encoding for five anchor-PNA probes with varying inter-anchor-PNA distances to characterize nanopore system's longitudinal resolution.

FIG. 5 is a schematic illustration showing synthesis of the polymer-linked anchor-PNA/capture-PNA conjugate e.g., a dendron-linked PNA following a two-step reaction (i) Azide-Alkyne cycloaddition and (ii) Michael Addition.

FIG. 6 is a schematic showing the synthesis and assembly of an exemplary 2S hybridization complex for single-plex system

FIG. 7 is a schematic illustration of the synthesis procedure for an exemplary fishing probe (coupling the anchor and hook motifs) showing a PNA-PNA fishing probe as an example. The coupling reaction follows a two-step process: (i) Azide-Alkyne cycloaddition and (ii) Michael Addition.

FIG. 8 is a schematic showing validation of the system's ability to do location-encoded multiplex mRNA detection.

FIGS. 9A and 9B are schematic illustrations of exemplary system such as a Nanofisher Toolbox applied to the multiplexed detection of a miRNA panel for colorectal cancer diagnosis, with fishing probe 1 generating a distinct signal on the nanopore current-time trace indicating the signal ‘read’ direction.

FIGS. 10A-10C illustrate a typical solid-state nanopore (FIG. 10A) and nanopore sensing experiment (FIG. 10B) showing dsDNA transport through a nanopore with corresponding ionic current trace (FIG. 10C). By applying a voltage bias across the nanopore, negatively charged molecules like DNA can be electrophoretically threaded through generating current signals, related to the molecule's size, length, structure and other properties. As shown in FIG. 10C, dsDNA is initially freely diffusing in the electrolyte solution, and the current trace shows a constant baseline current (Panel i of FIG. 10C). When dsDNA comes in close proximity to the pore (within the capture volume of radius rp) (Panel ii of FIG. 10C), it is subjected to the high electric field and is electrophoretically translocated through the pore, resulting in a modulation of current. When the dsDNA passes through the pore and exits, current returns to the baseline (Panel iii of FIG. 10C). The black trace illustrates an example of a current perturbation (DNA translocation) which is characterized by dwell time or Δt (blue) and peak blockade current or ΔI (orange.)

FIGS. 11A-11D are schematics illustrating the structure of PNA and its binding modes to double-stranded DNA. FIG. 11A shows a simplified structure of a PNA: DNA duplex showing Watson-Crick base-pairing between complementary nucleobases. FIG. 11B shows various PNA binding modes to dsDNA including i. Triplex invasion, ii. Triplex formation, iii. Double duplex invasion and iv. Duplex invasion. FIG. 11C shows a tail-clamp PNA (also referred as bis-PNA). FIG. 11D shows the structure of γ-PNA (left), and γ-PNA with miniPEG modification at the γ carbon.

FIG. 12 is a workflow diagram illustrating the DNA carrier preparation process from bacterial (plasmid) amplification of a desired 9448 bp sequence to purification of high-quality linear dsDNA as carrier of information.

FIG. 13 is a representative gel electrophoresis image of the purified linear dsDNA carrier. Lane 1 is the DNA/HindIII DNA ladder. Lane 2 is the plasmid after plasmid extraction. Lane 3 is a purified and linearized plasmid which serves as the DNA carrier in a single-molecule sensor.

FIGS. 14A and 14B show the chemical structure (FIG. 14A) and electrospray-Mass spectrometry (ES-MS) spectrum in positive-ion mode (FIG. 14B) of an exemplary fluorescently labelled anchor-PNA probe, Cy5-PNA-NLS. Observed and calculated m/z values are in good agreement, presented as m/z observed [m/z calculated]: 1452.67 [(5,809.12+4)⁴⁺=1,453.28], 1162.34 [(5,809.12+5)⁵⁺=1,162.82], 968.78 [(5,809.12+6)⁶⁺=969.18], 830.53 [(5,809.12+7)⁷⁺=830.87], 726.84 [(5,809.12+8)⁸⁺=727.14], 646.19 [(5,809.12+9)⁹⁺=646.45], 581.67 [(5,809.12+10)¹⁰⁺=581.91].

FIG. 15 is a schematic representation of the workflow used to generate preliminary results validating PNA-NLS invasion into dsDNA using gel shift assay (enhanced with fluorescent labeling). Briefly, the carrier DNA is enzymatically cut to produce a small fragment containing the PNA invasion target site. This fragment is purified then mixed with the PNA-NLS-Cy5 or biotin-PEG-γPNA¹-NLS probe for invasion.

FIG. 16 is a representative gel image showing successful invasion of PNA-NLS into the dsDNA carrier. The “target DNA” contains the sequence complementary to the PNA-NLS while the “Non-target DNA” does not contain any complementary sequence to the PNA-NLS. PNA-NLS-Cy5 is shown in red while the DNA stained by SYBR dye is shown in green. By fluorescently labeling the PNA-NLS, successful invasion is validated in two ways. Firstly, the co-localization of the red bands (representing PNA) and green bands (representing DNA) verifies the PNA-NLS invasion into the target DNA. This contrasts with the non-target DNA lanes where only green bands are seen. Secondly, the shift of the band with PNA-NLS compared to that without PNA-NLS also demonstrates successful invasion. Notably, the shift is greater when PNA-NLS is fluorescently labeled with Cy5 as expected due to its larger size.

FIG. 17 is a representative gel image showing γPNA-NLS sequence specific invasion into short linear DNA fragment.

FIGS. 18A-18C are representative gel images of XNA-based anchor motif invasion at different temperatures: 60 °C (FIG. 18A), 55 °C (FIG. 18B), and 50 °C. (FIG. 18C). When heated at 60 °C as shown in FIG. 18A, DNA forms a higher order of conformation around 400 bp size, even when the XNA-based probe is not present. When the incubation temperature is at and below 55 °C (FIGS. 18B and 18C), the 400 bp conformation is not present and the invasion band (DNA/probe complex) appears at around 300 bp size.

FIG. 19 is a representative image showing the effect of varying KCl concentrations on the invaded XNA-dsDNA complex. PNA invasion is performed at 55 °C for 2 hours. Following the invasion reaction, an 0.1 mol/L, 0.2 mol/L or 1.0 mol/L of KCl was added and the sample was incubated overnight at 4 °C. Gel electrophoresis was performed to determine if DNA/γPNA-NLS complex was still present.

FIGS. 20A-20D show the chemical structures and ES-MS spectrums in positive-ion mode of γPNA¹-NLS (FIGS. 20A and 20B) and biotin-PEG-γPNA¹-NLS (FIGS. 20C and 20D). Observed and calculated m/z values are in good agreement, presented by m/z observed [m/z calculated]: For γPNA¹-NLS, 1,145.31[(5,722.17+5)⁵⁺=1,145.43], 954.59[(5,722.17+6)⁶⁺=954.69], [818.36[5,722.17+7)⁷⁺=818.45], 715.94[(5,722.17+8)⁸⁺=716.27], 636.73[(5,722.17+9)⁹⁺=636.79]; and for biotin-PEG2-γPNA¹-NLS, 1492.91[(5,967.83+4)⁴⁺=1,492.96], 1194.53[(5,967.83+5)⁵⁺=1,194.56], 995.61[(5,967.83+6)⁶⁺=995.64], 853.52[(5,967.83+7)⁷⁺=853.54], 746.96[(5,967.83+8)⁸⁺=746.97], respectively.

FIG. 21 is a CD (Circular dichroism) spectra of conventional PNA with NLS (PNA¹-NLS) and ^{L}S-γPNA with NLS (γPNA¹-NLS) with a series of concentrations. All PNA samples were prepared in nuclease-free water at the indicated concentrations.

FIG. 22 is a representative gel shift image showing the successful invasion of XNA anchor motif (i.e., the γPNA¹-NLS) into the dsDNA carrier. The “191 bp DNA” contains the sequence complementary to the γPNA-NLS while the “268 bp DNA” does not contain any complementary sequence to the γPNA-NLS. Successful invasion is validated by the appearance of a new shifted band (indicated the invasion complex) with γPNA-NLS compared to that without γPNA-NLS or with the non-target DNA sequences.

FIG. 23 are representative images showing combination of the bifunctional probe with the 191 bp fragment of the carrier DNA and the model biomarker (SAPE). When the bifunctional probe is combined with the 191 bp fragment of the carrier DNA and the model biomarker (SAPE), a distinctive band appears in the electrophoresis gel at a much larger molecular weight compared to the bare DNA fragment. This band is absent in all controls and is indicative of the formation of a DNA-bifunctional probe-biomarker complex on the DNA carrier, and the successful binding of SAPE onto the bifunctional probe. The band also shows a fluorescence signal in both channels, that associated with the SYBR gold staining of DNA and the R-PE channel which is indicative of SAPE presence. Note that to confirm there is no bleed through between the two channels, we imaged the gel prior to SYBR gold staining in the R-PE channel (left, red). This shows the same band pattern as the gel after SYBR gold staining (right, red) which confirms that the signals observed are SAPE-specific and not due to spectral overlap.

FIGS. 24A and 24B are schematics of additional strategies for validation of anchor-PNA invasion into carrier dsDNA. Schematic illustration of the first strategy (FIG. 24A) where a biotinylated PNA-NLS probe will be used for dsDNA invasion. Capture of DNA (through the invasion complex) on streptavidin-coated beads would be used to validate successful invasion and analyze its efficiency. Schematic illustration of a second strategy (FIG. 24B) whereby the invaded sequence will be designed to overlap with a restriction enzyme recognition sequence. Successful PNA-NLS invasion can be validated through enzymatic digestion evident through the formation of a distinct band pattern in gel electrophoresis.

FIG. 25 is a schematic of an exemplary protocol for the preparation of the disclosed compositions and systems for the single molecule sensing method-DNA optical mapping.

FIG. 26 is a schematic of the application of the disclosed compositions and systems in a nanochannel array.

### DETAILED DESCRIPTION OF THE INVENTION

Poor prognosis and high mortality rates of many cancer types have been linked to late-stage diagnosis, when the disease is metastatic and less responsive to treatment.51,52 Apart from late-stage detection, most current diagnostic methods are invasive, expensive and non-specific, rendering them impractical for patients and the economy. Liquid biopsy tests, based on the detection of disease biomarkers from minute volumes of biological fluids, can transform the way diseases and pathogens are detected and managed. Among the various liquid biopsy biomarkers for cancer, miRNA has recently emerged as being highly sensitive and specific, but current gold-standard strategies to detect them are lacking, being complex, expensive, time-consuming while offering limited multiplexing. The disclosed compositions, systems and methods address this unmet clinical need and introduce a solution to the identified technological gap, being simple, versatile, sensitive, high-throughput and potentially amenable with point-of-care devices.

### I. DEFINITIONS

The term “amplification” as used herein refers to increasing the number of copies of a nucleic acid molecule, such as a gene or fragment of a gene, for example at least a portion of a biomarker RNA. The products of an amplification reaction are called amplification products. An example of in vitro amplification is loop-mediated isothermal amplification.

The term “functional” as used herein refers to a molecule having two binding sites to two different binding partners.

As used herein, the term “biomarker” refers to a molecular, histologic, radiographic, and/or a physiologic characteristic that is measured as an indicator of normal biological processes, pathogenic processes, or responses to an exposure or intervention, including therapeutic interventions. The biomarker may be a diagnostic and/or or prognostic biomarker used for detecting a tissue state or disease, monitoring a tissue state or disease, and/or predicting tissue state or disease. For example, a measure of a biomolecule that is a biomarker may provide information about a tissue state as a diagnostic biomarker, as well as information about future changes in the tissue state as a prognostic biomarker. Other examples of biomarkers include body mass index (BMI) as a physiologic biomarker, or tissue elasticity as a radiographic biomarker, both of which may be diagnostic biomarkers and prognostic biomarkers.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3” is complementary to the sequence “3′-T-C-A-S′.” Certain bases not commonly found in naturally occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence and can also be a cDNA.

The term “conditions sufficient for” as used herein in connection with the disclosed methods, refers to any environment that permits the desired activity, for example, that permits specific binding or hybridization between two nucleic acid molecules or that permits reverse transcription and/or amplification of a nucleic acid. Such an environment may include, but is not limited to, particular incubation conditions (such as time and/or temperature) or presence and/or concentration of particular factors, for example in a solution (such as buffer(s), salt(s), metal ion(s), detergent(s), nucleotide(s), enzyme(s), etc.).

The term “contact” as used herein in connection with the disclosed methods refers to placement in direct physical association; for example, in solid and/or liquid form. For example, contacting can occur in vitro with one or more primers and/or probes and a biological sample (such as a sample including nucleic acids) in solution.

As used herein, the term “sample” refers to in vitro as well as clinical samples obtained from a patient. In preferred embodiments, a sample is obtained from a biological source (i.e., a “biological sample”), such as tissue or bodily fluid collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material), nasopharyngeal aspirate, nasopharyngeal swab, throat swab, and other discussed herein and otherwise known in the art.

“Sensitivity” as used herein, is a measure of ability of a detection assay to detect the presence of a target sequence directly or indirectly (e.g., an analyte sequence) in a sample.

“Specificity,” as used herein, is a measure of the ability of a detection assay to distinguish a truly occurring target sequence (e.g., an analyte sequence) from other closely related sequences (e.g., other human-causing diseases and pathogens). It is the ability to avoid false positive detections.

The terms “target nucleic acid” or “target sequence” or “target segment” as used herein refer to a nucleic acid sequence of interest to be detected and/or quantified in the sample to be analyzed. Target nucleic acid may be composed of segments of a genome, a complete gene with or without intergenic sequence, segments or portions of a gene with or without intergenic sequence, or sequence of nucleic acids to which probes or primers are designed to hybridize. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

“Covalent linkage”, refers to a bond or organic moiety that covalently links molecules (e.g. fusion proteins) to a non-cellular surface.

As used herein, the term “polypeptide” refers to a chain of amino acids of any length, regardless of modification (e.g., phosphorylation or glycosylation).

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “host cell” refers to prokaryotic and eukaryotic cells into which a recombinant expression vector can be introduced.

As used herein, “transformed” and “transfected” encompass the introduction of a nucleic acid (e.g., a vector) into a cell by a number of techniques known in the art.

### II. COMPOSITIONS AND SYSTEMS

The disclosed compositions and devices enable sensitive and multiplex detection of biomarkers such as miRNA biomarkers without the use of expensive enzymes, complex amplification strategies, or multiple fluorophores for multiplexing. It is also isothermal, i.e. not requiring thermal cycling as is standard with traditional strategies based on qPCR. Thus, the disclosed system and compositions do not rely on enzymes such as CRISPR/cas.

The disclosed compositions can be used to detect one or more biomarkers which is used to code a carrier DNA sequence at intervals, by employing a XNA or other molecule complementary to the analyte of interest able to invade the DNA helix and bind to its complementary sequence i.e., the code.

The disclosed compositions and methods provide a xeno nucleic acid (XNA)-based method for multiplexed data encoding onto a DNA carrier which facilitates high-throughput decoding using ultrasensitive single molecule-based nanopore readout. A DNA molecule is decorated along its length—in a sequence-specific manner—with engineered bifunctional XNA-based probes at specific locations. The design, characteristics and properties of the engineered XNA probes enable the formation of a two-dimensional hybridization complex between the DNA carrier and target biomarker. These properties enable (i) the formation of a highly stable construct for robust information encoding: construct is stable across extreme physicochemical conditions of ionic strength and temperature, and (ii) simplicity, cost-effectiveness, and versatility in the data encoding strategy: strategy does not involve the use of enzymes or specific DNA sequences.

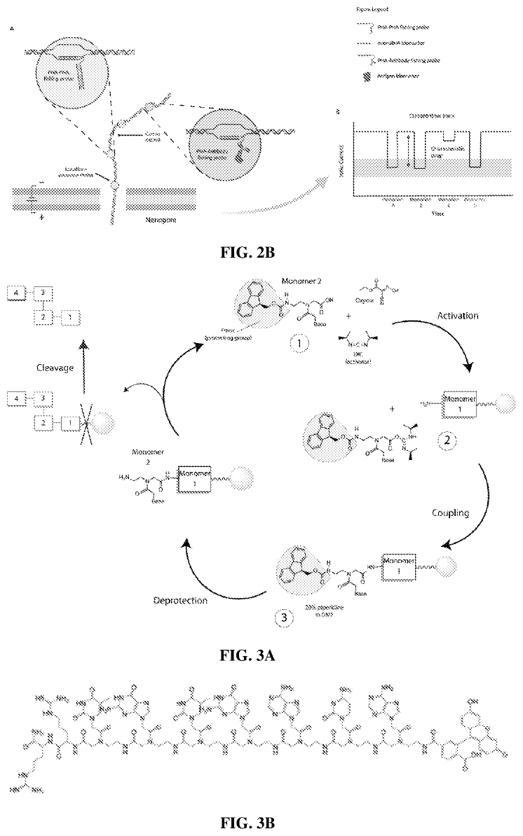

In some forms, multifunctional XNA-based probes are engineered such that one end invades and binds to a dsDNA carrier at sequence-specific pre-defined locations, while the other end acts as a probe for binding and capturing analytes of interest such as a target biomarker (FIG. 2). The nucleic acid constructs or complexes are designed so that their information can be decoded or read out by a single molecule sensing method e.g., a nanopore device or nanochannel in a high-throughput way. The location of the XNA probe invasion on the long DNA carrier encodes for the type of biomarker that the probe is targeting. After encoding data on the dsDNA-XNA hybrid constructs, the constructs subsequently get analyzed through a nanopore sensor which can directly decode multiplexed information based on their spatial position along the dsDNA carrier. For example, location 1 along the carrier encodes for biomarker 1, and so on.

The compositions include (i) a DNA carrier molecule comprising (decorated along its length—in a sequence-specific manner) sequences complementary to sequences in a Xeno-nucleic acid (XNA) based anchor and (ii) a bifunctional XNA-based probe. The bifunctional XNA-based probe includes two molecules separated by a linker: (1) a “XNA-based anchor” having a sequence complementary to a sequence on the DNA carrier; and (2) a “XNA-based hook” e.g., a bioreceptor specific to the analyte of interest. In some forms, the XNA in the XNA-based probe is a peptide nucleic acid (PNA). For example, in one embodiment, the XNA-based probe includes a first and a second PNA molecule separated by a linker.

The XNA-based probe in one embodiment includes a first and a second XNA-based molecule separated by a flexible linker. The XNA is preferably one with the capacity to invade a double stranded sequence. XNA-based probes are engineered such that one end invades and binds to a dsDNA carrier at sequence-specific pre-defined locations, while the other end acts as a probe e.g., nucleic acid binding partner or peptide binding partner for data capture (including target biomarker capture). The design, characteristics and properties of the engineered PNA probes enable the formation of a two-dimensional hybridization complex with the carrier DNA.

### A. DNA Carrier

The disclosed compositions include a DNA carrier molecule. DNA is preferred as the DNA carrier because: i. DNA is an inherent encoder of information with highly programmability and ii. the length and structure of DNA (including the existence of knots or probes) can be rapidly detected and read-out by single-molecule sensing technology e.g., nanopore technology.

Along its length, the DNA carrier molecule is decorated with one or more sequences complementary to sequences in a XNA-based probe at sequence specific defined locations. The dsDNA strand functions as a carrier of information, and this carrier will be spatially decorated along its length with XNA-based probes (also referred to as “fishing probes”) in a location encoded manner (FIG. 2B).

In some forms, the DNA carrier includes linearized DNA. Preferably, the linearized DNA is linearized plasmid DNA. In one exemplary form, the DNA carrier includes the 9448 bp sequence derived from the genome of the bacteriophage M13KO7. In another form, the DNA carrier includes linearized DNA derived from the genome of the bacteriophage M13SW8 (Wong, et al., Microb Cell Facts, 2023, 22:124).

Generally, any suitable dsDNA carrier can be used provided that the length of the dsDNA carrier is compatible with single-molecule sensing technologies e.g., nanopore sensors. In some forms, the DNA carrier is an adenovirus. Adenoviruses are large non-enveloped double stranded linear DNA viruses

The DNA carrier can have a size ranging from about 2.5 kbp to about 50 kbp. For example, the DNA carrier can have a size ranging from about 2.5 kbp to about 45 kbp, from about 2.5 kbp to about 40 kbp, from about 2.5 kbp to about 35 kbp, from about 2.5 kbp to about 30 kbp, from about 2.5 kbp to about 25 kbp, from about 2.5 kbp to about 30 kbp, from about 2.5 kbp to about 25 kbp, from about 2.5 kbp to about 20 kbp, from about 2.5 kbp to about 15 kbp, or from about 2.5 kbp to about 10 kbp. In some forms, the DNA carrier can have a size of about 2.5 kpb, about 5 kpb, about 10 kbp, about 15 kbp, about 20 kbp, about 25 kbp, about 30 kbp, about 35 kbp, about 40 kbp, about 45 kbp, or about 50 kbp.

### B. XNA-Based Probes

The compositions include a bifunctional XNA-based probe. Then number of XNA-based probes that can be accommodated on a DNA carrier depends on the length of the DNA carrier and the sensitivity of the analytical method such as the nanopore or nanochannel. Therefore, the maximum number of XNA-based probes that can be incorporated on a DNA carrier is partially dependent on the resolution of the single-molecule sensor e.g., nanopore or nanochannel, i.e., the minimum distance between two probes that can easily be distinguished. For example, the minimum distance between two probes on a solid state nanopore sensor is approximately about 600 bp to about 1000 bp. Thus, if the DNA carrier is about 10 kbp (which is generally be large enough to generate high signal-to-noise ratio for nanopore readout signals), then the maximum number of probes that can be incorporated on the DNA carrier is approximately 10000 bp/600 bp=16 probes. In some forms, the larger the size of the DNA carrier, the higher the number of XNA-based probes that can be incorporated on the DNA carrier. For example, a larger carrier DNA e.g., a 20 kbp DNA carrier may be able to accommodate double the number of XNA-based probes. However, if the analytical method has very high resolution, a first XNA-based probe can be located very close to a second XNA-based probe, such as about 400 bp apart. Preferably, for multiplex biosensing applications, a minimum of 5 biomarkers can be detected in one assay. In some forms, the distance between two XNA-based probes on the DNA carrier is from about 400 bp to about 1000 bp, from about 400 bp to about 900 bp, from about 400 bp to about 800 bp, from about 400 bp to about 700 bp, from about 400 bp to about 600 bp, or from about 400 bp to about 500 bp apart.

In some forms, DNA carrier has one XNA-based probe along its length. Preferably, the DNA carrier has more than one XNA-based probe along its length. In some forms, the DNA carrier has two, three, four, five, six, seven, eight, nine, ten, or more XNA-based probes along its length.

The single-molecular sensing device also limit the maximum number of codes. For nanopore sensing, carriers below 2.5 kbp will pass through the nanopore too quickly, limiting sensitivity and performance. For optical DNA mapping, the length is limited by the length of the nanochannel.

The bifunctional XNA-based probe includes two molecules separated by a linker: (1) a “XNA-based anchor” having a sequence complementary to a sequence on the DNA carrier; and (2) a binding partner or XNA hook e.g., a bioreceptor specific to the analyte of interest. Thus, the XNA-based probes generally contains two motifs: an “anchor” motif based on PNA probes with invasion capacity that can bind the carrier at specific sites and a polymer-linked “hook” motif which is capable of detect target biomarkers. The two motifs will be orthogonally linked to one another through a polymer linker through copper-free ‘click’ chemistry due to its speed, biocompatibility, bioorthogonal nature, and versatility as demonstrated by our prior work.

### 1. XNA-Based Anchor

The XNA-based probe includes a XNA-based anchor, which facilitates invasion of the dsDNA carrier at location-encoded sites on the dsDNA carrier. The XNA-based anchor e.g., a PNA-based anchor, is capable of invading mixed sequences with both purine and pyrimidine bases. Thus, preferably, the XNA-based anchor is designed such that duplex invasion can occur at any desired site along the dsDNA, with no sequence constraints.

In preferred forms, the XNA in the XNA-based anchor is a PNA. In some forms, the XNA is a preorganized PNA such as γ-PNA, which is modified at the γ-C atoms (through methyl, lysine, or PEG modifications). While this γ modification enables duplex invasion and significantly higher thermal stability when binding to target oligonucleotides compared with conventional PNA. In some forms, the anchor contains the sequence K-AAATAAATACTTATAGCAAAAA-K (SEQ ID NO:6). Other exemplary PNAs sequences include homopyrimidine PNAs e.g., Lys-NH-CCTCTCCTCCCT-CONH2 (SEQ ID NO: 27; Guanine (G)-rich PNAs e.g., H-KGGGTGGGGK-NH₂ (SEQ ID NO:28); and conjugates of PNAs with short peptides e.g., GGGLLVYGGGK (SEQ ID NO:29).

In some forms, the PNA is a pseudo-complementary PNA. In pseudo-complementary PNAs (pcPNAs), the canonical bases A and T are substituted with 2,6-diaminopurines and thiouracils. The 2,6-diaminopurines form a complementary pair with thymine through three hydrogen bonds whereas, 2-thiouracil (represented as U^{s} or ^{s}U) binds to Adenine with two hydrogen bonds. Unlike the A-T pair which only forms two hydrogen bonds, 2,6-diaminopurines can form 3 hydrogen bonds like C-G pair, which can increase the binding stability. pcPNA strongly binds with its complementary DNA (involving conventional A/T), since both D-T and A-Us pairs are sufficiently stable. Accordingly, a pair of complementary pcPNA strands are capable of efficiently invading a variety of sequences in dsDNA. Pseudo-complementary PNAs and their mechanisms of function are reviewed in detail in Liang, et al., BCSJ, 94 (6): 1737-1756 (2021), which is incorporated herein by reference in its entirety. For example, Izvolsky, et al., Biochemistry, 39:10908-10913 (2000) demonstrate the use of the pcPNAs: HLys₂-^{s}UCDDDCD^{s}UGC-LysNH₂ (SEQ ID NO:18); H-GCD^{s}UG^{s}U^{s}U^{s}UGD-LysNH₂ (SEQ ID NO:19); and HLys₂-^{s}U^{S}UGD^{S}UCDD-LysNH₂ (SEQ ID NO:20) to sequence-specifically target dsDNA at sites of mixed purine-pyrimidine sequences forming stable double-duplex invasion complexes (SU represents 2-thiouracil). In another example, Komiyama, et al., Nat. Protoc. 3:646 (2008), provides a protocol for generating pcPNAs, including H-(Lys) GU^{s}U^{s}DCU^{s}GDU^{s}GDU^{s}GDD(Lys)₂-NH₂ (SEQ ID NO:21); H-(Lys)CDU^{s}CDGU^{s}DDCCCGU^{s}D(Lys)₂-NH₂; H-(Lys)U^{s}GGCUsGCCDDGCCGU^{s}(Lys)₂-NH₂ (SEQ ID NO:22); H-(Lys)U^{s}U^{s}GGCDGCCDGU^{s}CCG(Lys)₂-NH₂ (SEQ ID NO:23); H-(Lys)U^{s}TACU^{s}GDTGD(Lys)-NH₂ (SEQ ID NO:24); and H-(Lys)U^{s}CAU^{s}CDGTAD(Lys)-NH₂ (SEQ ID NO:25) (U^{s} represents 2-thiouracil). In other forms, the XNA includes a nuclear localization signal (NLS), which is a structural motif of the nuclear localization protein, and facilitates conventional PNA to invade dsDNA in duplex invasion mode with high stability and invasion efficiency. A suitable XNA-based anchor is the PNA-NLS. PNA-NLA anchor-PNAs offer high thermostability, duplex invasion at any sequence, while maintaining greater simplicity and cost-effectiveness compared to γ-PNA. An exemplary nuclear localization signal is the nuclear localization signal of SV40, having the sequence PKKKRKV (SEQ ID NO:26).

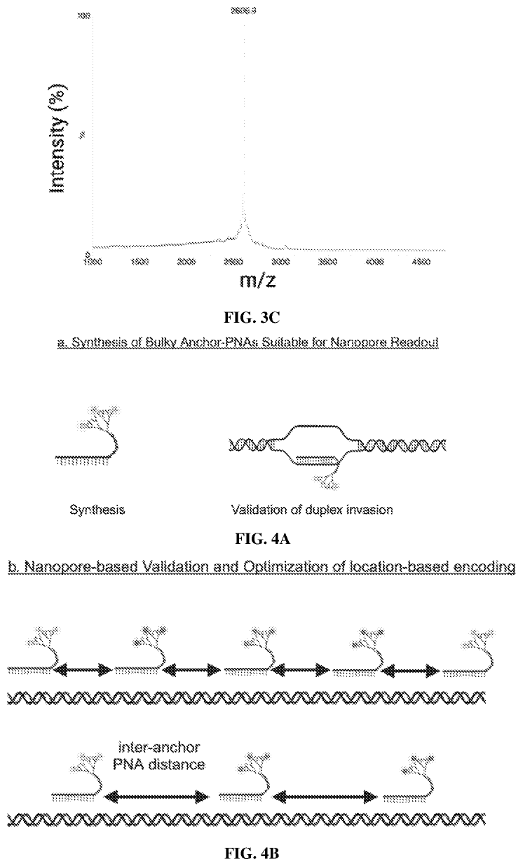

In some forms, the N-end of the XNA can be functionalized with a fluorescent tag. An exemplary XNA-probe with a fluorescent tag is an anchor-PNA probe with an NLS motif and a Cy5 fluorescent tag (see FIG. 3A).

In some forms, the XNA can be a 1,5-Anhydrohexitol nucleic acid (HNA), Cyclohexene nucleic acid (CeNA), Threose nucleic acid (TNA), Glycol nucleic acid (GNA), Locked nucleic acid, or an Fluoroarabino nucleic acid (FANA). For example, in some forms the XNA in the XNA-based probe is a locked nucleic acid. Locked nucleic acids (LNAs) are analog derivatives of RNA 2′-O and the 4′-C in ribonucleotide units are covalently linked by a methylene group. As a result, LNA/DNA and LNA/RNA duplexes are stable (Tm>60 °C) due to less entropy loss, when target sequence is longer than 10-nt. LNAs demonstrate increased hybridization affinity and specificity as a monomer or as a constituent of an oligonucleotide (Liang, et al., BCSJ, 94 (6): 1737-1756 (2021)). Sau, et al., Org. Biomol Chem., 8 (9): 2028-1036 (2010) describes double stranded 2′-N-(pyren-1-yl)methyl-2′-amino-α-L-LNA oligonucleotides (referred to as invader LNAs) for targeting short double stranded DNA. For example, Sau and colleagues tested and reported the hybridization data for several double stranded invader oligonucleotide probes, wherein each probe incorporated one or more LNA monomers, e.g., 5′-d(GGXATATATAGGC)-3′ (SEQ ID NO:31) and 3′-d(CCAXATATATCCG) (SEQ ID NO:32); 5′-d(GGTAXATATAGGC)-3′ (SEQ ID NO:33) and 3′-d(CCATAXATATCCG) (SEQ ID NO:34); 5′-d(GGTATATAXAGGC)-3′ (SEQ ID NO: 35) and 3′-d(CCATATAXATCCG) (SEQ ID NO:36), wherein X is the LNA monomer having the following structure:

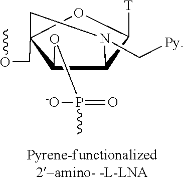

Generally, two factors influence the length of the XNA-based anchor: (i) minimum length to enable sequence-specificity (commonly 15-mer3) and (ii) ideal length for XNA e.g., PNA invasion which is dictated by the invasion strategy (typically from 16-mer to roughly 22-mer). In some forms, the length of the XNA-based anchor ranges from about 10-mer to about 30-mer, from about 12-mer to about 30-mer, from about 16-mer to about 30-mer, from about 19-mer to about 30-mer, from about 21-mer to about 30-mer, or from about 25-mer to about 30-mer. For example, the length of the nucleic acid binding partner ranges from about 16-mer to about 22-mer.

### 2. Analyte Binding Partners

The disclosed XNA-based probe includes binding partner (i.e., a hook) for the analyte of interest, which is connected to the XNA-based anchor via a linker. In some forms, the binding partner is a natural molecule. In some forms, the binding partner is a synthetic molecule. The binding partner can be a nucleic acid binding partner, a peptide binding partner, a protein binding partner, nanobody, a polysaccharide bioreceptor.

### a. Nucleic Acid Binding Partners

In some forms, the binding partner is a nucleic acid binding partner which is functionalized to bind a nucleic acid-based analyte such as microRNA, cell-free DNA, mRNA, and ribosomal RNA. Exemplary nucleic acid molecules that can be functionalized and included on the XNA-based probe include but are not limited to peptide nucleic acids (PNAs), other XNAs, and nucleic acid aptamers.

### i. Xeno Nucleic Acids

Synthetic mimics of natural nucleic acids (DNA/RNA), known as xeno nucleic acids (XNAs), can demonstrate superior biomedical performance to their natural analogues. Among them, peptide nucleic acid (PNA) probes represent a class of XNA oligos that combine the characteristics of peptides and nucleic acids. Thus, in some forms, the binding partner incorporated on the XNA-based probe is a peptide nucleic acid.

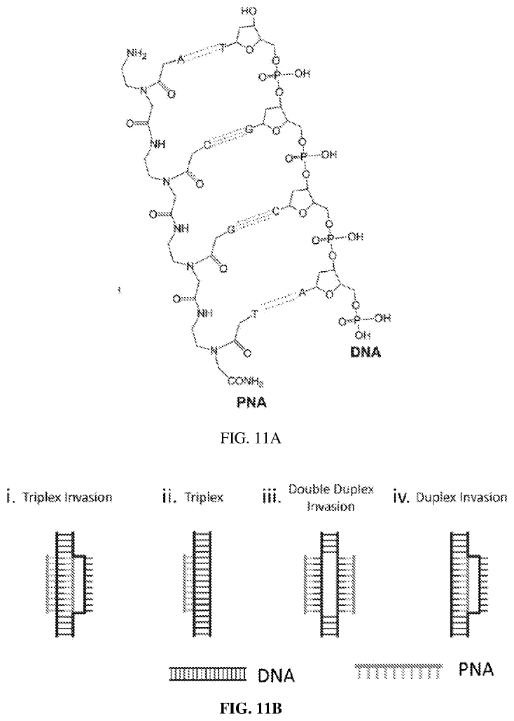

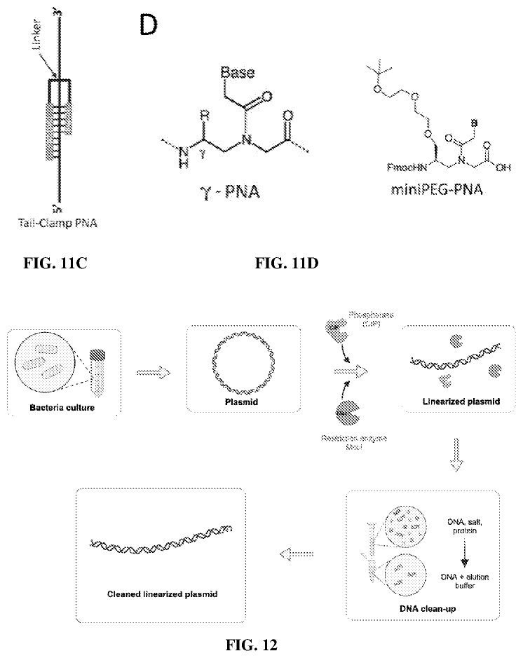

Peptide nucleic acids (PNAs) are synthetic pseudo-peptide oligomers analogous to standard oligonucleotides (or deoxy-oligonucleotides) but in which the sugar-phosphate backbone is replaced with a polyamide backbone.²¹ Instead of DNA's sugar-phosphate backbone, PNA is characterized by a neutral polyamide backbone consisting of N-(2-aminoethyl)glycine units (FIG. 11A). These probes can bind to complementary DNA, RNA or other nucleic acid through Watson-Crick and/or Hoogsteen base pairing rules (FIG. 11A). Due to their neutral charge, PNAs can bind to target nucleic acids with higher affinity and greater thermal stability than their natural oligonucleotide analogues, even at very low salt concentrations (Wang, J. L. Curr. Issues Mol. Biol. 1999, 1). They also offer greater sequence-specificity towards target sequences (Shakeel, S, et al., J. Chem. Technol. Biotechnol. 2006, 81, 892-899). Furthermore, their structural differences mean that they are highly stable across a wide range of temperatures and pHs (Uhlmann, E., et al., Angew. Chem. Int. Ed. 1998, 37, 2796-2823), and additionally resist enzymatic degradation, which is highly valuable for biological applications (Demidov, V., et al., Biochem. Pharmacol. 1994, 48, 1310-1313). Unlike natural nucleic acids, PNAs can also be engineered to invade dsDNA in a sequence-specific manner through Watson-Crick base pairing.

The ability of PNA to invade dsDNA in a sequence-specific manner allows for rational design of a system for multiplexed information encoding based on the location of the PNA-based probe along the dsDNA carrier. A distinctive feature of PNA is its ability to invade dsDNA through a variety of invasion or binding modes as highlighted in FIG. 11B. PNAs can mimic DNA triplex invasion^{47, 48}. In this mode, PNA can invade into dsDNA by breaking the original hydrogen bonds of Watson-Crick base pairing (FIG. 11B, panel i). This mode requires the target DNA strand to be homopurine or homopyrimidine, and usually a tail-clamp PNA or bisPNA is used to enhance binding stability (FIG. 11C).

Another mode of invasion which also requires the target to be homopurine or homopyrimidine is the triplex mode (FIG. 11B, panel ii). In this mode, PNA does not invade the double-strand but binds to it through Hoogsteen base pairing, thus offering significantly lower stability compared to Watson-Crick base pairing⁴⁹. In double duplex invasion mode (FIG. 11B, panel iii), two individual strands of PNA bind with complementary dsDNA at the same time through Watson-Crick base-pairing at any target sequence desired. In order to prevent self-binding of the two PNAs, one PNA contains unconventional nucleobases (e.g. 2,6-diaminopurine (D) and 2-thiouracil (Us)).

Another invasion mode in DNA-based data encoding applications is the duplex invasion mode (FIG. 3B, iv). To enable this type of invasion, PNA with a variety of chemical modifications have been developed, including modifications at the α or γ position on the PNA backbone (FIG. 3D), or conjugation of standard PNA with functional peptides at the N or C-terminus.

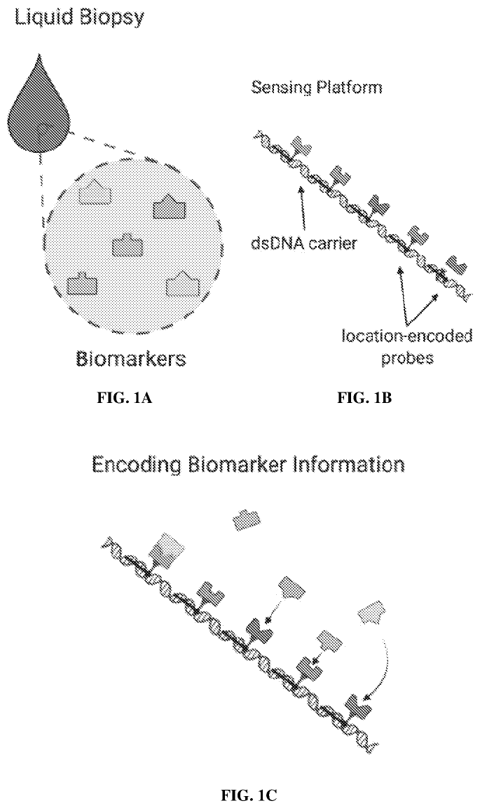

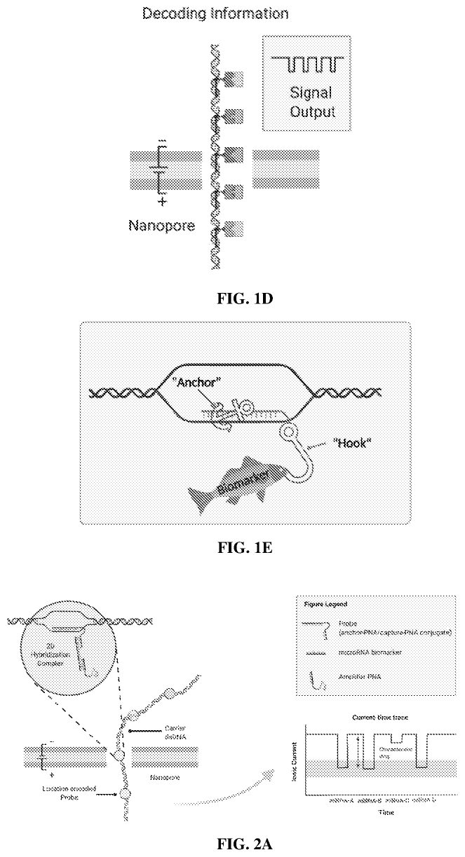

The PNA sequences and dendron structures will be synthesized using standard solid-phase peptide synthesis (SPPS)²⁵ techniques and based optimized protocols from prior work (FIG. 1) (Al Sulaiman, D, et al., Angew. Chem. Int. Ed Engl. 2017, 56, 5247-5251; Al Sulaiman, D., et al., ACS Nano 2019, 13). Preferably, the ‘click’ chemistry reaction will be used for the polymer conjugation reactions due to its speed, biocompatibility, bioorthogonal nature (Kim, E et al., Chem. Sci. 2019, 10, 7835-7851; Northrop, B., et al., Polym. Chem. 2015, 6, 3415-3430), and versatility as demonstrated by our prior work (Al Sulaiman, D., et al., ACS Nano 2019, 13). FIGS. 3A-3C is a schematic showing the methodology of solid phase peptide synthesis (SPPS) used for PNA and peptide synthesis. In an exemplary form, the compositions include (i) a DNA carrier molecule comprising (decorated along its length—in a sequence-specific manner) with sequences complementary to sequences in the PNA based anchor and (ii) bifunctional PNA-based probe which include complementary on the one end to the sequences the analytes of interest and complementary on the other end to sequences on the DNA carrier, the complementarity being provided by a first and a second PNA molecule. Thus, the PNA-based anchor in one embodiment includes a first and a second PNA molecules separated by a flexible linker. The PNA is preferably one with a double strand invading PNA. PNA-based probes are engineered such that one end invades and binds to a dsDNA carrier at sequence-specific pre-defined locations, while the other end acts as a probe for data capture (including target biomarker capture). The design, characteristics and properties of the engineered PNA probes enable the formation of a two-dimensional hybridization complex with the carrier DNA.

In some forms, the length of the PNA binding partner ranges from about 8-mer to about 25-mer, from about 12-mer to about 25-mer, from about 16-mer to about 25-mer, from about 19-mer to about 25-mer, or from about 21-mer to about 25-mer. For example, the length of the PNA binding partner ranges from about 16-mer to about 18-mer.

### ii. Nucleic Acid Aptamers

The binding partner in the XNA-based probe can be a nucleic acid aptamer. Nucleic acid aptamers are typically oligonucleotides ranging from 15-50 bases in length that fold into defined secondary and tertiary structures, such as stem-loops or G-quartets. The oligonucleotide may be DNA or RNA and may be modified for stability. A nucleic acid aptamer generally has higher specificity and affinity to a target molecule than an antibody. Nucleic acid aptamers preferably bind the target molecule with a Kd less than 10-6, 10-8, 10-10, or 10-12. Nucleic acid aptamers can also bind the target molecule with a very high degree of specificity. It is preferred that the nucleic acid aptamers have a Kd with the target molecule at least 10, 100, 1000, 10,000, or 100,000-fold lower than the Kd with other molecules. In addition, the number of target amino acid residues necessary for aptamer binding may be smaller than that of an antibody.

Nucleic acid aptamers are typically isolated from complex libraries of synthetic oligonucleotides by an iterative process of adsorption, recovery and reamplification. For example, nucleic acid aptamers may be prepared using the SELEX (Systematic Evolution of Ligands by Exponential Enrichment) method. The SELEX method involves selecting an RNA molecule bound to a target molecule from an RNA pool composed of RNA molecules each having random sequence regions and primer-binding regions at both ends thereof, amplifying the recovered RNA molecule via RT-PCR, performing transcription using the obtained cDNA molecule as a template, and using the resultant as an RNA pool for the subsequent procedure. Such a procedure is repeated several times to several tens of times to select RNA with a stronger ability to bind to a target molecule. The base sequence lengths of the random sequence region and the primer binding region are not particularly limited. In general, the random sequence region contains about 20 to 80 bases and the primer binding region contains about 15 to 40 bases. The specificity to a target molecule may be enhanced by prospectively mixing molecules similar to the target molecule with RNA pools and using a pool containing RNA molecules that did not bind to the molecule of interest. An RNA molecule that was obtained as a final product by such technique is used as an RNA aptamer.

Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698. An aptamer database containing comprehensive sequence information on aptamers and unnatural ribozymes that have been generated by in vitro selection methods is available at aptamer.icmb.utexas.edu.

In some embodiments, the nucleic acid aptamer may contain one or more modified nucleic acids (also referred to as xeno nucleic acids, or XNAs) for added chemical functionalities that may increase binding affinity of the nucleic acid aptamer to the immuno-complex. Non-limiting modified nucleic acids include but are not limited to unnatural base pairs (UBPs), base modifications such as for example, C7-modified deaza-adenine, C7-modified deaza-gaunosine, C7-modified deaza-cytosine, C7-modified deaza-uridine; and sugar modifications such as for example, ribulonucleic acid, α-L-threose nucleic acid (TNA), 3′-2′ phosphonomethyl-threosyl nucleic acid (tPhoNA) and 2′-deoxyxylonucleic acid (dXNA). In some forms, the modified nucleic acid may be introduced in the nucleic acid aptamer by in vitro evolution using an alternative for the phosphodiester backbone such as for example, phosphorothioates, boranophosphate, phosphonate, alkyl phosphonate nucleic acid, and peptide nucleic acid. In some forms, the modified nucleic acid may be introduced in the nucleic acid aptamer via a mutant T7 RNA polymerase that is tolerant of substitutions at the 2′ position of the furanose ring. Substitutions that may be attached to C2′ include but are not limited to a fluorine, an amine, or a methoxy group. In some forms, the modified nucleic acid may be introduced in the nucleic acid aptamer via R-group modifications at the 5th position of uracil. In these forms, the R-group can be one of many different sidechains known to those of skill in the art, ranging from hydrophobic to hydrophilic. Incorporation of synthetic nucleotides into nucleic acid aptamers using phosphodiester replacements and modified bases are known to those of skill in the art (See for example, Mayer G. Angew Chem Int Ed Engl. (2009) 48: pages 2672-2689; Keefe, A. D. and Cload, S. T. Curr Opin Chem Biol. (2008); 12: pages 448-456; Appella, D. H. Curr. Opin. Chem. Biol. (2009) 13 (5-6): pages 687-696).

For example, Cao H-Y, et al. BMC Cancer. 2014; 14:699 describes a cy-apt 20 aptamer having the sequence: CGACCCGGCACAAACCCAGAACCATATACACGATCATTAGTCTCCTGGGCCG (SEQ ID NO:4), labeled with fluorescein isothiocyanate (FITC) detects gastric cancer in AGS human gastric adenocarcinoma cells with the emittance of strong fluorescence intensity. Exemplary nucleic acid aptamers that can be incorporated into the XNA-based probe as the binding partner include nucleic acid aptamers for detecting cancers and/or neurodegenerative disorders. Suitable nucleic acid aptamers for a variety of cancers are described in Goh et al, Journal of Cancer, 2023; 14 (13): 2491-2516, which is incorporated herein by reference in its entirety.

### b. Peptide and Protein Binding Partners

In some forms, the binding partner is a peptide or protein binding partner, which is functionalized to bind a peptide-based or protein analyte such as hormones, enzymes, neuropeptides, antimicrobial peptides, and toxins. Exemplary peptides and proteins that can be used as the binding partner include but are not limited to antibodies, nanobodies, and peptide aptamers.

### i. Antibodies

In some embodiments, the XNA-based probe can include an antibody, as the binding partner for example. In some forms, the antibody binds to a cancer antigen, e.g., tumor-associated antigens or proteins linked to oncogenic processes. For example, the antibody may recognize antigens expressed on tumor cells, such as carcinoembryonic antigen (CEA) or HER2/neu. In some forms, the antibody exhibits specificity for cancer-specific antigens that are pivotal in the growth, proliferation, or metastatic activity of cancer cells. Such antigens include, but are not limited to, the epidermal growth factor receptor (EGFR), known for its role in various forms of cancer including lung and colorectal cancers, or the prostate-specific membrane antigen (PSMA), which is primarily associated with prostate cancer.

Antibodies that can be used include whole immunoglobulin (i.e., an intact antibody, such as IgA, IgD, IgE, IgG, and IgM, and including subclasses or isotypes, e.g., IgG1, IgG2, IgG3, IgG4, IgA1 and IgA2). Suitable antibodies encompass, but are not limited to, monoclonal antibodies, polyclonal antibodies, chimeric antibodies, bispecific antibodies, multispecific antibodies, heteroconjugate antibodies, mutants thereof, humanized antibodies, human antibodies, and any other modified configuration of the immunoglobulin molecule that includes an antigen recognition site of the required specificity,

In some embodiments, the antibody can be a humanized antibody. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

Native antibodies, and some other antigen binding proteins including some non-antibody antibody fragments and fusion proteins, can be heterotetrameric glycoproteins having two identical light chains and two identical heavy chains. Other antigen binding proteins including some non-antibody antibody fragments and fusions proteins may have few or more domains. Each heavy chain has at one end a variable domain (“VH”) followed by a number of constant domains (“CH”). Each light chain has a variable domain at one end (“VL”) and a constant domain (“CL”) at its other end. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each contain four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the antibodies typically contain at least the CDRs necessary to recognize and/or bind a target antigen (e.g., a tumor or cancer antigen). Non-antibody antigen binding proteins such as antibody fragments and fusion proteins may also include at least the CDRs necessary to recognize and/or bind a target antigen (e.g., a tumor or cancer antigen).

In some embodiments, the antibodies or non-antibody antigen binding proteins such as antibody fragments and fusion proteins may include one of each of a heavy chain CDR1, CDR2, and CDR3 in combination with one of each of a light chain CDR1, CDR2, and CDR3.

Anti-cancer antibodies (e.g., used for detection and/or neutralization) are known in the art and are suitable for use herein. For example, suitable antibodies include commercially available antibodies such as MA1-101, MA5-15837, MA5-32025, MA1-099, and MA5-35327 from ThermoFisher Scientific.

Anti-cancer antibodies are also described in U.S. Pat. Nos. 7,943,743 and 11,390,674 which are both hereby incorporated by reference in their entirety. Any antibody or antibody fragment disclosed in U.S. Pat. Nos. 7,943,743 and 11,390,674 can be used. For example, U.S. Pat. No. 7,943,743 describes human monoclonal antibodies to programmed death ligand 1 (PD-L1). PD-L1 expression has been found in several murine and human cancers, including human lung, ovarian and colon carcinoma, and various myelomas (Iwai et al. (2002) PNAS 99:12293-7; Ohigashi et al. (2005) Clin Cancer Res 11:2947-53).

In another example, Prostate-Specific Antigen (PSA) is a chymotrypsin-like serine protease (of the kallikrein family) produced by the prostate epithelium, and is abundant in seminal fluid. PSA can be detected in the sera of patients with prostatic carcinoma and is used to confirm prostatic acinar cell origin in primary and metastatic carcinoma and to rule out non-prostatic carcinoma mimics. An anti-prostate specific antigen antibody can be used to detect PSA. Anti-PSA antibodies are commercially available and can be obtained from ThermoFischer Scientific e.g., PSA Recombinant Rabbit Monoclonal Antibody (4H23L24) (Catalog No. 701640); PSA Monoclonal Antibody (A67-B/E3), Biotin (Catalog No. MA1-10447); PSA Monoclonal Antibody (A67-B/E3) (Catalog No. MA1-19217); and KLK3/PSA Monoclonal Antibody (4A1E2) (Catalog No. 60338-1-IG).

A second exemplary analyte that could be recognized and bound is CD63 (LAMP-3, lysosome-associated membrane protein-3), a glycoprotein of tetraspanin family, the expression of which is used for predicting the prognosis in earlier stages of carcinomas. Diseases associated with CD63 dysfunction include melanoma and Hermansky-Pudlak Syndrome. An anti-CD63 antibody can be used as a binding partner for the detection of CD63, and therefore to detect early stage carcinoma e.g., melanoma. Anti-CD63 antibodies are commercially available and can be obtained from ThermoFisher Scientific e.g., CD63 Monoclonal Antibody (MEM-259) (Catalog No. MA1-19281), and CD63 Monoclonal Antibody (Ts63) (Catalog No. 10628D).

In some forms, the antibody binding partner recognizes and binds a hormone. An exemplary hormone that can be bound is Vitamin D, and an exemplary binding partner for Vitamin D is 25-OH Vitamin D3 antibody. 25-OH Vitamin D3 antibodies are commercially available such as from ThermoFisher Scientific e.g., 25-OH Vitamin D3 Recombinant Rabbit Monoclonal Antibody (RM3) (Catalog #MA5-27880); 25-OH Vitamin D3 Recombinant Rabbit Monoclonal Antibody (RMH04) (Catalog #MA5-44545); and 25-OH Vitamin D3 Recombinant Rabbit Monoclonal Antibody (RM428) (Catalog #MA5-44546); and from GenScript e.g., 25-OH VD3 (32F9C4) (Catalog #V00101).

In some forms, the antibody binding partner detects and binds growth hormone e.g., an anti-growth hormone antibody. Anti-growth hormone antibodies are commercially available such as from ThermoFisher Scientific e.g., Growth Hormone Recombinant Rabbit Monoclonal Antibody (JM108-09) (Catalog #MA5-32604); Growth Hormone Recombinant Rabbit Monoclonal Antibody (5E9) (Catalog #MA5-49842); and Growth Hormone Monoclonal Antibody (KT34) (Catalog #MA5-49588).

In some forms, the binding partner is a molecule that recognizes and binds a polysaccharide or a fragment of a polysaccharide e.g., a carbohydrate specific antibody. Carbohydrate-specific antibodies are widespread among all classes of immunoglobulins; thus, carbohydrate antigens eliciting an immune response represent structures consisting of monosaccharides and oligosaccharides that are foreign to the host (Sterner E, et al., ACS Chem Biol. 2016; 11:1773-83).

A large pool of serum IgM and IgG recognizes a variety of carbohydrate antigens (Kappler and Hennet, Genes & Immunity volume 21, pages 224-239 (2020)). These most prominently recognized antigens include the monosaccharides α-rhamnose, α-GlcNAc and β-GlcNAc, and the sulfated Gal(β1-4) GlcNAc structure (Bovin N, et al. Biochim Biophys Acta. 2012; 1820:1373-82). Antibodies against β4-linked oligosaccharides of Glc, α-Gal and GlcNAc(β1-4) GlcNAc are also commonly observed. The repertoires of carbohydrate antigens recognized show a large inter-individual variability among human beings. Despite the recognition of mono- and disaccharide epitopes, most circulating carbohydrate-specific antibodies bind with low specificity to larger glycoconjugates, thus preventing the occurrence of disseminated antibody-mediated inflammatory reactions and autoimmunity (Bovin N, et al. Biochim Biophys Acta. 2012; 1820:1373-82). A-Rhamnose is a monosaccharide antigen associated with high antibody titer in human serum. This prominence is explained by the absence of rhamnose on human glycoconjugates and its widespread occurrence on microbial polysaccharides. Another human xenoantigen associated with carbohydrate-specific antibodies is the sialic acid N-glycolylneuraminic acid (NeuGc). Through the inactivation of the cytidine-monophosphate-N-acetylneuraminic acid hydroxylase gene, humans have lost the ability to produce NeuGc besides NeuAc. The contact to glycoproteins containing NeuGc stimulates the production of high antibody titers toward NeuGc. Antibodies specific for NeuGc do not cross-react with NeuAc despite the close structural similarity between both sialic acids. Carbohydrate-specific antigens and antibodies are further reviewed in Kappler and Hennet, Genes & Immunity volume 21, pages 224-239 (2020).

### ii. Nanobodies

In some forms, the peptide binding partner for incorporation into the disclosed XNA-based probe are not whole antibodies, but more compact recognition domains such as a nanobody or a sybody. As used herein, non-antibody protein refers to a protein that is not a whole antibody which is a multidomain protein. By contrast, a nanobody, also known as a single domain antibody, is an antibody fragment consisting of a single monomeric variable antibody domain e.g., the antigen-specific domain (VHH) of antibodies obtained from camelids and dromedaries. Like a whole antibody, a nanobody is able to bind selectively to a specific antigen. With a molecular weight of only 12-15 kDa, single-domain antibodies are much smaller than common antibodies (150-160 kDa) which are composed of two heavy protein chains and two light chains, and are also smaller than Fab fragments (~50 kDa, one light chain and half a heavy chain) or single-chain variable fragments (scFv, ~25 kDa fusion of two variable domains, one from a light and one from a heavy chain).

Camelids naturally produce one class of antibodies composed only of heavy chains in which the target recognition module is composed of a single variable domain (VHH or Nb). (De Meyer, et al., 2014; Kubala, et al., 2010). The nanobody offers unique properties, simpler structural conformation and high stability in a range of different conditions (De Meyer, et al., 2014). The VHH of a camelid antibody can be obtained by genetic engineering to yield a small protein having high affinity for a target, resulting in a low molecular weight antibody-derived protein known as a “camelid nanobody”. See U.S. Pat. No. 5,759,808; Stijlemans, et al., 2004 J Biol Chem 279:1256-1261; Dumoulin, et al., 2003 Nature 424: 783-788; Pleschberger, et al. 2003 Bioconjugate Chem 14:440-448; Cortez-Retamozo, et al. 2002 Int J Cancer 89:456-62; and Lauwereys, et al. 1998 EMBO J. 17:3512-3520; each of which is incorporated by reference herein in its entirety. Engineered libraries of camelids antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the camelid nanobody can be “humanized”.

Camelid nanobodies have low molecular weights approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques.

In some forms, the XNA-based probe includes a nanobody, which binds to an antigen which indicates the presence of a disease e.g. cancer, or pathogen, e.g. virus or bacteria. An exemplary nanobody that can be incorporated in the XNA-based probed is an anti-PD-L1 nanobody, which recognizes and binds PD-L1 (Programmed death 1 ligand 1), a biomarker for many cancers e.g., melanoma, liver cancer, gastric cancer, renal cell carcinoma, ovarian cancer, colon cancer, breast cancer, esophageal cancer, head, and neck cancer. Exemplary anti-PD-L1 nanobodies are provided in U.S. Pat. No. 11,466,085, which is incorporated herein in its entirety, and include but are not limited to SEQ ID NO: 1 to SEQ ID NO:3 provided below. CDR1 is bolded, CDR2 is bolded and italicized, and CDR3 is italicized.

(SEQ ID NO: 1)
QVQLQESGGGSVQTGGSLRLSCAASTSLYSYNYMAWFSQAPGKGREGVAA
VYVGDGRPYYADSVKGRFTISLDSAKNAVYLQMNSLKPEDTAMYYCAAAP
GPLSHNYWYTSANYDYWGQGTQVTVSS;
(SEQ ID NO: 2)
QVQLQESGGGSVQAGGSLRLSCAVSRYSASNNVIKWMGWFRQAPGKEREG
VAALYTSGGNTYYADSVKGRFTISRDYSENTVSLQMNNLKPEDTGMYYCA
TTVGTVLAGPLSARKYNYWGQGTQVTVSS;

QVQLQESGGGSVQTGGSLRLSCAASTSLYSYNYMA WFSQAPGKGREGVAAVYVGD GRPYYADSVKGRFTISLDSAKNAVYLQMNSLKPEDTAMYYCAAAPGPLSHNYWYTSA NYDYWGQGTQVTVSS (SEQ ID NO:3). Other examples include, but at not limited to SARS 1/2 nanobody (VHH72), shown below: QVQLQESGGGLVQAGGSLRLSCAASGRTFSEYAMGWFRQAPGKEREFVATISWSGG STYYTDSVKGRFTISRDNAKNTVYLQMNSLKPDDTAVYYCAAAGLGTVVSEWDYD YDYWGQGTQVTVSSGS (SEQ ID NO: 17) The nanobody can be naturally-derived from immunization of an animal or synthetically-derived (i.e., a sybody) or a combination thereof. Sybodies are disclosed for example, in Zimmerman, et al., eLife, 2018; 7: e34317, and Deneka et al., Nature Communications, 12, Article number: 5435 (2021).

### iii. Peptide Aptamers

In some forms, the binding partner is a peptide aptamer. Peptide aptamers are small peptides with a randomized amino acid sequence that are selected for their ability to bind a target molecule. Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display, and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

Exemplary peptide aptamers that can be incorporated into the XNA-based probe as the binding partner include peptide aptamers for detecting cancers and/or neurodegenerative disorders. Suitable peptide aptamers for a variety of cancers are described in Goh et al, Journal of Cancer, 2023; 14 (13): 2491-2516, which is incorporated herein by reference in its entirety. In a second example, Ali, et al., Cell Mol. Life Sci, 2023 80 (6): 139 describes a peptide aptamer targeting Aβ-PrP-Fyn axis in a mouse model of Alzheimer's disease.

In some forms, the binding partner is a peptide aptamer suitable for binding carbohydrates such as glycans e.g., a glycan specific aptamer.

### iv. Other Proteins

In some forms, the binding partner is a protein that can bind to carbohydrates or polysaccharides e.g., glycans. Exemplary proteins that can bind to glycans include but are not limited to antibodies (described above), lectins, microbial adhesins, and viral agglutinins.

### Lectins

In some forms, the binding partner is a lectin. Lectins are carbohydrate-binding proteins that selectively recognize and reversibly bind to sugar groups that are part of other molecules. Exemplary carbohydrates to which lectins bind include but are not limited to d-fructose, d-galactose, 1-arabinose, d-xylose, d-mannose, d-glucose, d-glucosamine, d-galactosamine, 1-fucose, various uronic acids, sialic acid, and their combinations to form other di- and oligosaccharides, or other biomolecules.

Lectins are classified based on their structure or subcellular location. Division based on their location includes integral lectins located in membranes as structural components, or soluble lectins present in intra- and intercellular fluids, which can move freely.

Division according to lectin structure consists of several different types of lectins, such as C-type lectins (binding is Ca²⁺ dependent), I-type lectins (carbohydrate recognition domain is similar to immunoglobulins), galectin family (or S-type, which are thiol dependent), pentraxins (pentameric lectins) and P-type lectins (specific to glycoproteins containing mannose 6-phosphate) (Santos, et al., Curr. Top. Pept. Protein Res. 2014; 15:41-62).

Exemplary C-type lectin receptors that can serve as the binding partner include but are not limited to proteoglycans or lecticans such as aggrecan, brevican, neurocan, versican; and Prolectin (C-type lectin domain containing 17A). Exemplary L-type lectins that can serve as the binding partner include but are not limited to Calnexin (binding affinity for Non-reducing glucose residues in an oligosaccharide (Glc (Man) 9 (GlcNAc) 2); Lectin, mannose-binding 1 (affinity for α-(1-2) mannans with free OH-3, OH-4 and OH-6); and Calreticulin (Non-reducing glucose residues in an oligosaccharide (Glc (Man) 9 (GlcNAc) 2).

In some forms, the lectin is a chitolectin or chilectin e.g., chitinases and chitolectins, which recognizes and binds chitins. Chitinases are active proteins that bind and hydrolyze oligosaccharides and chitolectins are able to bind oligosaccharides but do not hydrolyze them (Dalal P., et al., Biochem. Biophys. Res. Commun. 2007; 359:221-226; Kilpatrick et al., Biochim. Biophys. Acta. 2002; 1572:187-197). Exemplary chitinases and chitolectins that can serve as the binding partner include but are not limited to Chitinase 3 like 1 and oviductin or oviductal glycoprotein 1 (recognizes and binds chitin); Chitinase 3 like 2 (recognizes and binds Chitooligosaccharides); and Stabilin-1 interacting chitinase-like protein (recognizes and binds N-acetylgalactosamine (GalNAc), N-Acetylglucosamine (GlcNAc), ribose, and mannose).

Additional lectins that can serve as the binding partner are reviewed in Raposo et al., Biomolecules, 11 (2): 188 (2021), which is incorporated herein by reference in its entirety.

### Microbial Adhesins

In some forms, the binding partner is a microbial adhesin. A microbial adhesin, typically a protein or glycoprotein, is a molecule found on the surface of microorganisms that enables them to adhere to host cells and tissues as well as to surfaces in their environment. A number of secreted bacterial toxins are capable of binding glycans. Bacterial toxins that can serve as the binding partner include but are not limited to crystal toxins; botulinum toxins (A-E) (receptor specificity for gangliosides GT1b and GQ1b); Clostridium difficile toxin A; tetanus toxin (derived from Clostridium tetani); and Escherichia coli heat-labile toxin (receptor specificity for GM1) (see Varki, A., Cummings, R. D., Esko, J. D., et al. (Eds.). (2022). Essentials of Glycobiology (4th ed.). Cold Spring Harbor Laboratory Press).

### 3. Linkers

The XNA-based anchor and the binding partner e.g., a nucleic acid probe or peptide binding partner are separated by a linker. The term “linker” as used herein includes, without limitation, peptide linkers. In some forms, the linker is a flexible linker, and is preferably selected to be stable under the reaction conditions e.g., a short linker.

In one embodiment, the linker is a polymeric linker e.g., polyethylene glycol. In other forms, the linker is a peptide linker such as a short, flexible peptide linker such as a serine or glycine based amino acid trimers and their mixtures. In some forms, the linker is an aliphatic chain such as hexanoic acid.

The linker can be any size provided it does not interfere with the binding of the epitope. The length of the linker can be optimized for minimal steric hinderance and maximum efficiency of binding between binding partner and the target biomarker. For example, monovalent single-chain antibody variable fragments (scFvs) in which the C-terminus of one variable domain are typically tethered to the N-terminus of the other variable domain via a 15 to 25 amino acid peptide or linker. Therefore, the number of linkers, composition of the linker(s), and length of the linker(s) can be determined based on the desired valency of the scFv as is known in the art. The linker(s) can allow for or drive formation of a di-, tri-, and other multivalent scFv.

In some embodiments, the linker includes one or more glycine and/or serine amino acid residues. For example, an exemplary linker can include 4-8 amino acids. In some forms, an exemplary linker includes the amino acid sequence GOSSRSS (SEQ ID NO:8). In some forms, a linker includes 15-20 amino acids, for example, 18 amino acids. For example, the linker includes the amino acid sequence GQSSRSSSGGGSSGGGGS (SEQ ID NO:9). Other flexible linkers include, but are not limited to, the amino acid sequences Gly-Ser, Gly-Ser-Gly-Ser (SEQ ID NO:10), Ala-Ser, Gly-Gly-Gly-Ser (SEQ ID NO:11), (Gly4-Ser)₂ (SEQ ID NO: 12) and (Gly4-Ser)₄ (SEQ ID NO:13), and (Gly-Gly-Gly-Gly-Ser)₃ (SEQ ID NO:14). Other exemplary linkers include, for example,

(SEQ ID NO: 15)
RADAAPGGGGSGGGGSGGGGS
and
(SEQ ID NO: 16)
ASTKGPSVFPLAPLESSGS.

### 4. Amplifier Binding Partner

In some forms, the system includes a third amplifier binding partner e.g., a PNA (herein amplifier PNA, labelled with the suitable label). When present, the amplifier PNA complementarily binds with target nucleic acid biomarker resulting in an increased signal-to-noise ratio in the nanopore or nanochannel readout. The length is mainly dependent on the biomarker target length or size and must ensure specificity. For a non-nucleic acid-based target, the amplifier may also be another bioreceptor, such as a secondary antibody or nanobody.

As demonstrated in the non-limiting Examples, an exemplary amplifier sequence for targeting miR-141 is TGTGACA.

Other amplifying binding partners include but are not limited to Streptavidin-biotin, dendrimer, dendrons, and star co-polymers.

### C. Kits

Also provided are kits containing some or all the materials of the compositions, and/or reagents needed to detect analytes such as biomarkers in a biological sample.

The kits generally include one or more dsDNA carrier molecules and one or more XNA-based probes. The XNA-based probes included in the kit contain one or more XNA-based anchors and one or more binding partners separated by a linker.

In some forms, the kit contains one or more buffer solutions e.g., hybridization buffers and/or washing buffers. A hybridization buffer can be included in the kit to promote the invasion of the XNA-based probe in the dsDNA carrier molecule. A washing buffer can be included in the kit to remove non-specifically bound substances from the system, thereby increasing the detection signal.

In some forms, the kit contains a positive and a negative control.

The kits can include instructions for use. For example, the kits can include detailed instructions for the proper use of the kit, including step-by-step procedures for preparing samples, adding reagents, and interpreting the results. In some forms, the kits include troubleshooting guidelines to assist users in resolving common issues that may arise during the use of the kit.

### III. METHOD OF MAKING

The disclosed compositions are designed to be “read out” or “decoded” using single-molecule detection technologies. Therefore, methods of making the DNA carrier and the XNA-based probes are also provided. The methods include instructions and considerations for designing and preparing the DNA carriers.

### A. Instructions and Considerations for Designing and Preparing DNA Carriers

To design a linear dsDNA strand which is suitable for single molecule sensing methods e.g., nanopore-based readout and multiplexed biomarker detection, certain criteria should be considered with regards to the DNA carrier length and its accurate synthesis or amplification to generate microgram amounts suitable for single molecule detection studies as discussed below.

### 1. Length Considerations for DNA Carrier

The DNA carrier length may be designed to ensure optimal signal-to-noise ratio during nanopore readout or decoding studies. In some forms, this is achieved by maximizing the dwell time or peak current of the DNA translocation event, both of which can be increased by increasing the DNA length²⁵. Generally, standard single-molecule sensing technologies e.g., solid state nanopore studies can reliably and accurately detect DNA ranging in lengths between 3.5 kbp³¹ to 48.5 kbp²⁴.

Another consideration is that the DNA carrier has adequate length to enable multiplexed data encoding, ensuring two neighboring anchor-PNA sites along the carrier result in separate distinguishable nanopore signals. This parameter is also called the longitudinal resolution of the biosensor. Studies with standard solid-state nanopores of 3.7 nm diameter have reported a resolution or minimum distance of 400 bp between neighboring probes³².

Based on the above-mentioned requirements, and assuming a longitudinal resolution double that found in the literature (i.e. 800 bp), the DNA carrier preferably has a length of about 10 kbp which would enable the detection of at least 10 distinct biomarkers on the same carrier, thus providing sufficient capacity for multiplexed biomarker detection in clinical applications. For example, miRNA biomarker panels of only three miRNA can achieve an area under the curve (AUC) greater than 0.9 (sensitivity=0.90, specificity=0.70) for the diagnosis of stages I, II, III, and IV colorectal cancer, while increasing the panel to five or six miRNA can achieve an AUC above 0.95 (sensitivity=0.91, specificity=0.91) for the diagnosis of stage I, II, III, and IV colorectal cancer^{16,17}.

### 2. Large-Scale Synthesis of DNA Carrier and its Sequence Validation

After designing the DNA carrier length, its sequence and origin may be considered. In one exemplary form, the 9448 bp sequence derived from the genome of the bacteriophage M13KO7 can be used. Any suitable phage sequence can be used as long as it satisfies the length requirements defined in part (a) above while being of non-human origin.

To produce sufficient amounts of the required sequence at high purity (preferably the scale of 10s of micrograms), bacterial culture (E. coli) can be used for amplification after purchasing the plasmid in bacterial stab format (e.g., from Addgene). Microgram amounts of DNA can be produced using this method following existing protocols⁵⁴.

After amplification, the plasmid can be extracted from the bacteria through commercial extraction kits (e.g., QIAGEN plasmid Kits) followed by linearization using restriction enzymes MscI and alkaline phosphatase QuickCIP. Finally, commercial DNA purification kits (e.g., QIAEX II system kit), can be used to remove any enzymes or salts which may interfere with the disclosed XNA-based probe invasion efficiency or future single-molecule detection studies.

A flow diagram summarizing the abovementioned steps is provided in FIG. 12. Validation of amplification and successful linearization can be conducted through gel electrophoresis studies compared to a DNA ladder. The sequence of the amplified DNA carrier can also be confirmed by a third generation sequencing method, e.g., Oxford Nanopore Technologies (ONT).

### B. Instructions and Considerations for Designing and Synthesizing XNA-Based Probes

The design, characteristics, and properties of the engineered XNA-based probes enable the formation of a two-dimensional complex between the carrier DNA and target analyte e.g., a target biomarker.

### 1. Design of the XNA-Based Anchor

Methods for establishing an exemplary XNA-based anchor motif, e.g., a PNA-anchor, with the ability to invade the dsDNA carrier at specific locations along the sequence, are provided.

Methods of synthesizing invader PNAs are known and take advantage of PNA's ability to perform duplex invasion. Some of the reported invader PNAs can only target specific sequences⁵⁶. However, the presently described anchor-PNAs can be designed such that duplex invasion can occur at any desired site along the target, with no sequence constraints, i.e. anchor-PNA can invade mixed sequences⁵⁵ with both purine and pyrimidine bases). Two major classes of PNAs that can perform duplex invasion in this way^{32, 56}.

An exemplary method uses preorganized PNA such as γ-PNA, which is modified at the γ-C atoms via methyl, lysine, or PEG modifications. The γ modification facilitates duplex invasion and significantly higher thermal stability when binding to target oligonucleotides compared with conventional PNA, although a potential limitation includes high cost and challenges to produce or source commercially.

Another method to facilitate duplex invasion involves covalently linking a positively charged peptide to conventional PNA⁵⁶. Among such peptides, the nuclear localization signal (NLS), which is a structural motif of the nuclear localization protein⁵⁷ can help conventional PNA to invade dsDNA in duplex invasion mode with high stability and invasion efficiency. Due to the existence of the NLS peptide, the PNA-NLS complex can enter the nucleus, along with either an enzyme or plasmid, thereby exhibiting great potential for other therapeutic applications of the disclosed XNA-based probes⁵⁸.

Considering the above, PNA-NLS was investigated as one exemplary design (or version 1) of the disclosed XNA anchors e.g., anchor PNAs as it offered high thermostability, duplex invasion at any sequence, while maintaining greater simplicity and cost-effectiveness compared to γ-PNA⁴⁸. Knowing the carrier sequence, XNA anchors e.g., anchor-PNAs can be rationally designed to invade or decorate the DNA carrier at any desired site.

Another consideration when designing anchor-PNA is its nucleotide length to ensure maximum sequence specificity (i.e. anchor-PNA must invade the carrier at one desired location); thus, a minimum sequence length of about 16 nucleotides is chosen for exemplary designs.

### 2. Synthesis of XNA-Based Anchor

The designed anchor-PNAs can be synthesized via automated Fmoc-based solid-phase peptide synthesis (SPPS) based on previously optimized protocols on Chorus Purepep Synthesizer (Gyros Protein Technologies). Briefly, the PNA sequences may be synthesized on a resin (e.g. Rink-Amide MBHA resin) using a PurePep™Chorus automated peptide synthesizer. Synthesis may be carried out using a standard FMOC solid-phase synthesis protocol, including systematically repeated steps of coupling and deprotection interspaced with washings (3×2 mL dimethylformamide (DMF)). Coupling may be conducted as a double cycle using 4 equivalents of FMOC-amino acid and DIC/Oxyma Pure as coupling reagents in DMF (4 ml) at 85 °C for 5 minutes. Deprotection may be achieved by two cycles of 20% piperidine in DMF (3 ml) at 65 °C for 40 seconds.

PNA may then be cleaved from the resin using a cleavage cocktail of TFA (Trifluoroacetic acid), Triisopropyl silane (TIPS) and H₂O at a ratio of about 95% volume to 2.5% volume to 2.5% volume at room temperature for about 2.5 hours. The resin may then be removed by filtration, and the filtrate can be precipitated in ice-cold diethyl ether. The precipitate can then be isolated via centrifugation (4000 rpm, 10 minutes, 4 °C), dissolved in acetonitrile/water at a ratio of 70:30 v/v %) and then freeze-dried.

The crude PNA product may then be purified by semi-preparative reversed-phase high-performance liquid chromatography on Shimadzu LC-20 HPLC system equipped a C18 column (5 μm, 30×250 mm). Eluents that can be used include 0.1% formic acid in H2O (A) and 0.1% formic acid in MeCN (B). Semi-preparative runs can be performed by applying a linear gradient (at 35 mL/min) of 10% to 80% B over 30 minutes.

The N-end of the anchor-PNA can be functionalized with a fluorescent tag using the manual approach of SPPS in a syringe reactor, as illustrated in FIG. 3A. After cleavage of the PNA from the SPPS resin, the product can be purified and characterized using semi-preparative HPLC and LCMS systems, respectively, optimized for PNA probe analysis.

### 3. Validation of Location-Based Encoding Capacity of the Anchor-PNA

Invasion conditions can be optimized by studying various physicochemical parameters including temperature, duration, buffer type, and concentration. For example, invasion conditions can be based on 5 mM HEPES buffer (pH 7.0), at 70 °C, for a duration of 1.5 hours⁵⁹. Other conditions can also be investigated including invasion buffer ionic strength, composition, and the ratio of anchor-PNA to dsDNA.

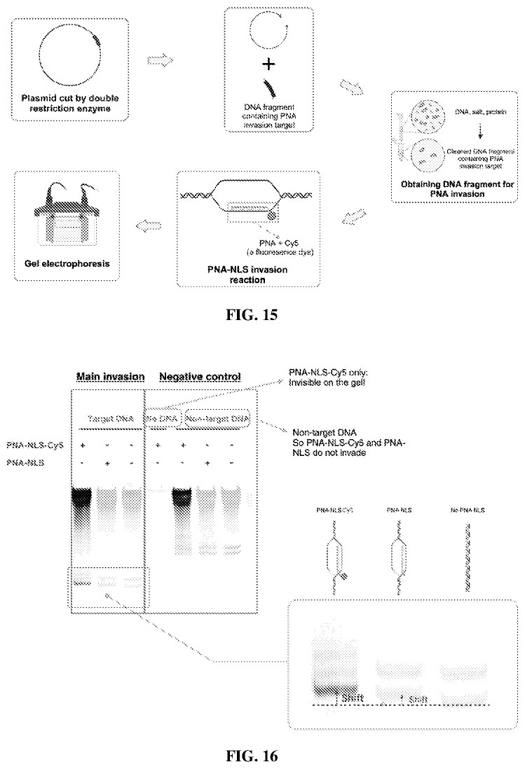

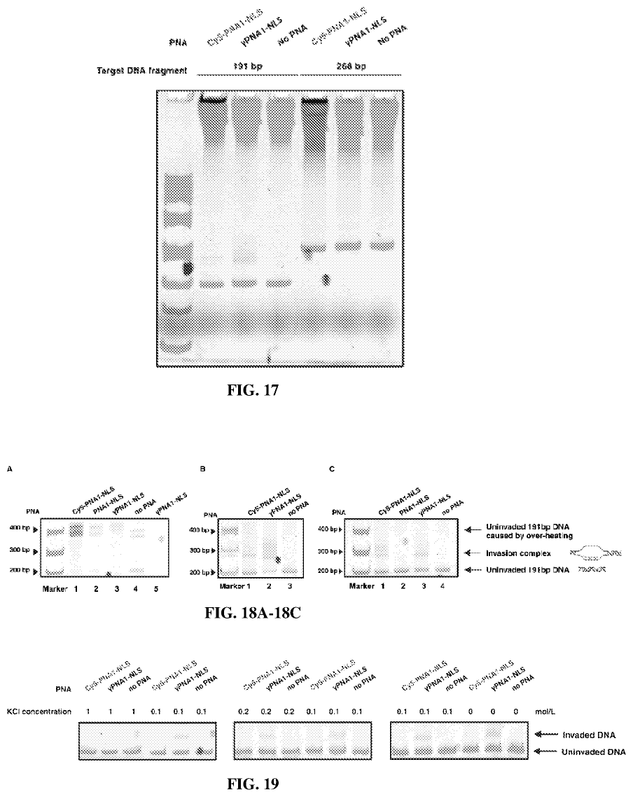

To promote PNA invasion and stability, a lower ionic strength and the use of salts such as NaCl, LiCl, or MgCl₂ can be considered⁵⁶. Successful invasion can be validated using a number of biochemical/optical assays e.g., with gel shift assays as illustrated in FIG. 15. In some forms, the PNA: DNA invasion complex is expected to have a lower electrophoretic mobility, compared to the smaller non-invaded or bare DNA carrier, resulting in a shift in its band. However, since the DNA carrier size (~10 kbp) is significantly larger than the PNA size (16 mer), the DNA carrier can be enzymatically cut into a smaller fragment (e.g., about 300 bp, surrounding the target site) to increase the PNA: DNA size ratio thus enhancing the gel shift compared to uninvaded DNA (see Methodology workflow in FIG. 15).

In some forms, tagging the anchor-PNA with a fluorophore e.g., Cy5 (red) dye can provide additional validation of invasion during the gel shift assay (i.e. gel shift assay enhanced with fluorescent labeling). In addition to mobility shift observed through electrophoresis, the invaded DNA can be expected to also display a red fluorescent signal in addition to a green signal from SYBR staining.

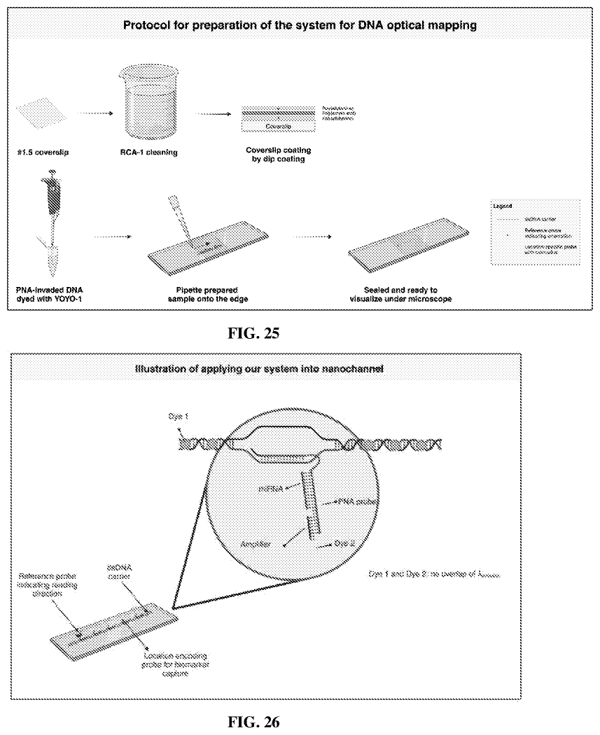

In addition to the conventional strategies for validating invasion, two additional methodologies can be used to validate invasion (see FIGS. 25A and 24B). One method is a biotin-streptavidin-based strategy relying on the interaction between biotin-tagged anchor-PNA and streptavidin-coated beads, wherein the detection of DNA in the bead elution solution would indicate successful invasion (FIG. 24A).

The second method is an enzyme-based strategy relying on PNA's resistance to enzymatic degradation. In this method, the invasion of PNA-NLS into the dsDNA would impede restriction enzyme digestion when a restriction enzyme recognition sequence overlaps or partially overlaps with the PNA-NLS target sequence (FIG. 24B). In this way, successful invasion could be validated by detecting a distinct band pattern compared to the non-invaded DNA band pattern after restriction enzyme digestion.

### 4. Validation of the Anchor-PNA Invasion Capacity

a. Synthesis of Bulky Anchor-PNAs Suitable for Nanopore Readout

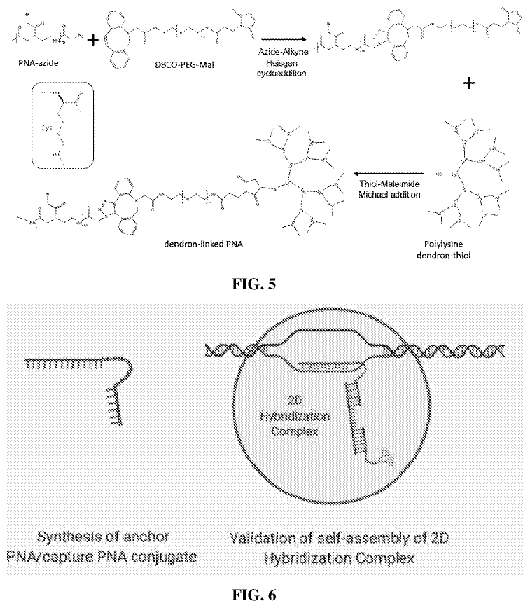

Provided is a new approach to facilitate nanopore detection of the PNA: DNA invasion complex. The anchor-PNA probe can be tagged with a bulky and rigid molecule (e.g., a polylysine dendron⁶⁰), in addition to being labelled with fluorescent tags to facilitate validation. To prepare this dendron-linked PNA, the anchor-PNA motif can be extended with a linker using a heterobifunctional polymer of DBCO-PEG-Mal, and then conjugated to the dendron of poly(lysine) with a thiol core and acetylated amine-ends (FIG. 5).

To prevent non-specific interactions between the probes and nanopore, the poly(lysine)amine-ends can be acetylated resulting in an overall neutral charge. To achieve optimal fluorescence for the validation studies, two ways for fluorophore tagging on the dendron can be studied and compared: i. a single fluorophore can be incorporated in the dendron core, and ii. a number of fluorophores can be conjugated on the dendron surface using its amine-ends. Synthesis can be validated via HPLC and mass spectrometry, whereas successful invasion of the disclosed design for the anchor-PNA can be validated as previously described and via nanopore studies.

b. Nanopore-Based Validation of Location-Based Encoding

Using the synthesized bulky anchor-PNA, single-molecule studies e.g., nanopore studies can be conducted to prove invasion at a single site by comparing the ionic current traces e.g., nanopore traces generated from blank carrier DNA versus anchor-PNA-invaded carrier DNA (FIG. 4A).

Next, single-molecule sensing technology e.g., nanopore technology can be used to validate the toolbox's location-based data encoding capacity. In an exemplary method, four more bulky anchor-PNAs (total of 5) can be first prepared and designed to have varying inter-anchor-PNA distance to characterize the nanopore system's longitudinal resolution (FIG. 4B). An exemplary starting distance of 400 bp can be used based on similar studies³². This distance can then be increased until the nanopore system is able to detect two distinct nanopore signals (corresponding to neighboring probes).

### 5. Design and Preparation of the XNA Binding Partner

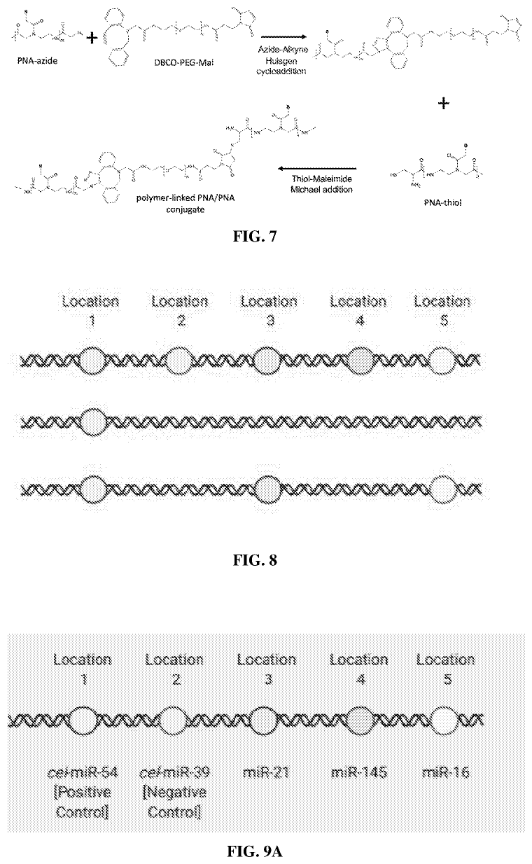

In some forms, to prepare the entire XNA-binding partner or fishing probe (with an anchor-hook 2D design), the anchor-PNA can be synthesized with an azido-end functionality, while the binding partner or the hook motif can be functionalized with a cysteine residue, providing a thiol functionality. For example, a PNA-PNA fishing probe can be designed. The two motifs can be covalently linked together to form a XNA-based binding partner or fishing probe, using the heterobifunctional polymer of α-dibenzocyclooctyne-@-maleimide polyethylene glycol (DBCO-PEG-Mal), through the orthogonal chemical reactions of copper-free Azide-alkyne Huisgen cycloaddition and thiol-maleimide coupling (FIG. 7).

In some forms, the linker length can be varied to optimize capture or biomarker “fishing” efficiency without affecting invasion efficiency. The insights gained from the design and synthesis of the initial PNA-PNA fishing probe can be used for the development of constructs with other types of bioreceptors such as antibodies and aptamers for protein biomarker detection. To attach the hook motif in these fishing probes, the same chemistry of thiol-maleimide coupling will be used exploiting cysteine-engineered antibodies⁶¹ and thiol-modified DNA aptamers⁶².

### 6. Validating the Biomarker Detection Capacity

The disclosed compositions can be designed and generated to detect not only nucleic acids such as microRNA, but also other clinically-relevant biomarkers e.g., proteins.

a. Preparation of a Nanofisher Toolbox for Multiplexed Detection of a miRNA Panel

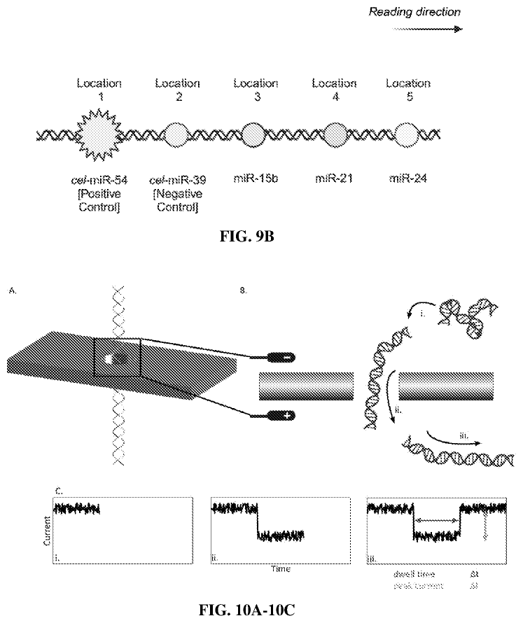

In an exemplary form, the disclosed compositions can be used for the detection of a clinically-relevant miRNA panel for colorectal cancer diagnosis. For example, five PNA-PNA fishing probes can be designed and generated to target: 1 positive control miRNA (cel-miR-54), 1 negative control miRNA (cel-miR-39), and 3 endogenous miRNAs which are known to be dysregulated in colorectal cancer patients: miR-15b, miR-21 and miR-24.¹⁷ For example, miR-15b⁶⁵ and miR-21⁶⁶ are significantly upregulated in colorectal cancer patients compared to healthy controls. Conversely, miR-24 is downregulated in colorectal cancer patients. The probes can be designed to bind the DNA carrier at specific locations with an inter-probe distance. Furthermore, the first fishing probe-indicating location 1 on the carrier—must generate a characteristic signal (compared to the other probes) in order to signal the read direction. To address this, that fishing probe 1 must contain a larger bulky moiety (e.g. larger dendron) to distinguish it from signals generated by other probes on the nanopore readout current trace. This location will also be chosen to encode the positive control biomarker (FIG. 9B).

For example, miR-141 is a candidate biomarker of colorectal cancer and prostate cancer. The sequence for the target hsa-miR-141 is 5′AACACUGUCUGGUAAAGAUGG3′ (SEQ ID NO: 30). The sequence of the fishing probe ‘hook’ motif e.g., a 16-mer PNA probe can be designed and synthesized via SPPS to be complementary to the bolded and italicized miRNA sequence. To validate successful biomarker capture by the hook motif, the target miR-141 can be purchased with a fluorescent tag and binding assays with the fishing probe-encoded DNA carrier can be conducted and optimized. In this way, validation studies may be based on gel shift assays and gel electrophoresis studies enhanced by fluorescence imaging for example.

b. Multiplexed Detection of a Protein Biomarker Panel

The disclosed compositions can be applied to the detection of non-nucleic acid biomarkers, e.g., protein-based biomarkers following the considerations and protocols outlined above. For example, antibodies or peptide aptamers can be used as the hook motifs in the XNA-based probes. A number of fluorescently labeled antigens can be purchased commercially along with their corresponding antibodies. The antibodies can be incorporated into the XNA-based probe (i.e., the fishing probe) as the hook motif through the chemistry previously described. For example, cysteine-engineered antibodies can be clicked to maleimide-functionalized linkers as shown in FIG. 2B. In other forms, peptide aptamers can also be incorporated as the hook motif in the XNA-base probe. Validation studies can be conducted as described above using fluorescence-based assays, gel electrophoresis assays and nanopore studies (NUS).

### IV. METHOD OF USING

Also disclosed are methods of using the disclosed compositions for detecting an analyte such as a biomarker in a sample. The disclosed compositions are suitable for use with single molecule sensing platforms e.g. nanopores or nanochannel arrays.

Typically, the method includes (1) collecting a biological sample to be tested for one or more analytes, and (2) contacting the biological sample with a plurality of the DNA carriers and a plurality of the XNA-based probes for detection of presence or absence of the analyte.

### A. Single Molecule Sensing Nanotechnologies

### 1. Solid-State Nanopores for High-Throughput Biomarker Information Decoding

Label-free, single-molecule sensing nanotechnologies represent ideal candidates for biomedical applications that rely on the detection of low copy number of rare biomolecules in small volumes of biofluid samples (Justin, G. J., et al., Angew. Chem. Int. Ed. 2016, 55, 11354-11366). Examples include solid-state nanopore sensors. The technology measures changes in current as analytes are transported (translocated) across nanopores are particularly useful with the disclosed compositions. a nanometer-sized aperture. Compared to biological nanopores, synthetic nanopores developed from solid-state materials such as silicon dioxide (SiO2) (Storm, A. J. et al., Nat. Mater. 2003, 2, 537), or silicon nitride (SiN), present powerful, more robust, scalable, and cheaper sensing platforms for label-free detection of biomolecules (Tang, Z.; et al., Nanomaterials and Nanotechnology 2016, 6). Compared to conventional bulk assays, nanopores can rapidly analyze individual dsDNA molecules at sub-picomolar concentrations, while a large number of readouts provide high accuracy.

The disclosed system preferably does not include a single stranded DNA or a single stranded RNA as a carrier molecule, nor does it include an enzyme such as any enzyme from the CRISPR/Cas family of enzymes (dead or live), for example, dCas9, or their accessory carrier molecules such as a cas-specific guide sequence.

Thus, the disclosed systems in a preferred embodiment includes the disclosed DNA carrier molecule, PNA anchors and a nanopore sensor, preferably a solid state nanopore sensor. Compared with biological nanopores, solid-state nanopores are mechanically robust and durable with a tunable pore size and geometry. Solid-state nanopores with sizes as small as 1.3 nm have been fabricated in various films using engraving techniques, such as focused ion beam (FIB) and focused electron beam (FEB) drilling methods. Typical fabrication technologies for solid-state nanopores is reviewed in Chen, et al., Sensors (Basel). 2019 April; 19 (8): 1886. doi: 10.3390/s19081886.

Structurally, a nanopore consists of a nanometric constriction or aperture separating two chambers filled with an electrolyte solution, such as KCl or LiCl. By applying an electric potential across the chambers, one can electrophoretically drive charged molecules like DNA and proteins across the pore. Translocation of the charged molecule then generates modulations in pore conductance which can be measured and analyzed as the nanopore signal (ionic current trace)²⁵. By analyzing the ionic current signals including the translocation peak current and dwell time (FIG. 10C), information encoded in the DNA can be decoded. The two major classes of nanopores are biological and synthetic (solid-state) nanopores. Compared to biological nanopores which are primarily derived from natural channel proteins, solid-state nanopores offer greater stability, robustness, versatility and suitability for long-term device usage. Furthermore, the synthetic alternatives can be produced at lower cost and with greater efficiency²¹ from materials like silicon nitride, graphene, or molybdenum sulfide.^{21,22} Due to their resilience against extreme physicochemical conditions, solid-state nanopores represent more robust alternatives, suitable across a wide range of applications.²¹ Consequently, they have been investigated and implemented as high-throughput sensing and analysis technologies for a range of biomolecules, including viral RNAs²⁶ and proteins,²⁷ can be used.

### 2. Nanochannel Arrays for Optical Read-Outs

In addition to electrical-based single-molecule sensing using nanopores, the disclosed compositions are suitable for read-out using optical-based single-molecule sensing. For example, the carrier DNA and XNA-based probes can be labelled with different fluorescent molecules, with single-molecule sensing conducted via fluorescence microscopy.

In some forms, optical DNA mapping can be used to visualize large DNA molecules as they are stretched either by shear forces or through nanochannels, then visualized at single-molecular level by using fluorescence microscopy. In some forms, to stretch the DNA molecules, nanochannel arrays are applied, which stretch the DNA molecules in the nanometric constriction channel (with a cross-section of around 100 nm×150 nm), usually by gas or buffer solution flow. By loading the fluorescently labelled DNA onto the nanochannel arrays, individual molecules of DNA can be directly visualized via optical fluorescence microscopy, from which the DNA carrier and the code pattern (as produced from the XNA-based probe) can be obtained from the recorded image. (reviewed in Levy, S. L., & Craighead, H. G. (2010), Chemical Society reviews, 39 (3), 1133-1152)

In one exemplary form, binding of the target biomarker may introduce a fluorescent label at a specific position along the DNA carrier (for example via the amplifier binding partner described above). The label may have a different emission wavelength compared to the fluorescent molecule staining the carrier DNA. For example, DNA can be stained using YOYO-1 dye and binding of the target biomarker would introduce a Cy-5 dye (which has a non-overlapping emission wavelength). In this way the location of the Cy-5 dye along the DNA carrier would signify the target biomarker detected (see FIGS. 25 and 26).

### B. Analytes to be Detected

The disclosed compositions and systems are used to detect analyte(s) of interest present in a sample, such as a sample obtained from a biological sample, including but not limited to blood, plasma, tissue, and other bodily fluids. The method includes contacting sample with a composition containing a carrier DNA and/or one or more XNA-based probes and optionally, a XNA amplifier. The disclosed method further comprises detecting the positions of the XNA-based probe in the sample using single molecule sensing technology e.g., a nanopore sensor as described above. The methods and systems are multiplexed in that the carrier DNA can be designed to bind multiple XNA-based probes, where each XNA-based probe binds to a different analyte of interest, in the sample. For example, the system can include more than 2 XNA-based probes, for example, 3, 4, 5, 6, 7, 8, 9, 10, etc.

Analytes of interest which can be detected using the disclosed compositions, systems, and methods, include but are not limited to biomarkers such as nucleic acid biomarkers e.g., microRNA, cell-free DNA, mRNA, and ribosomal RNA; proteins e.g., hormones, enzymes, antigens, and antibodies; polysaccharides, viral particles, and other analytes of clinical and environmental interest.

### C. Controls

The methods including analyzing one or more analytes and/or biomarkers typically includes comparing to a control. For example, the level of a biomarker detected in a sample obtained from the subject can be compared to a control. Suitable controls will be known to one of skill in the art. Controls can include, for example, standards obtained from healthy subjects, such as subjects without the disease or disorder, or non-diseased tissue from the same subject. A control can be a single or pooled or averaged values of like individuals using the same assay. Reference indices can be established by using subjects that have been diagnosed with the disease or disorder with different known disease severities or prognoses.

The disclosed compositions and methods can be further understood through the following examples.

### EXAMPLES

The overall aim of the project was to develop and validate a high-throughput platform for sensitive and multiplex detection of miRNA biomarkers derived from minute volumes of biofluid samples using a nanopore-based approach (Scheme 1). The aim is for the sensing platform to translate rare and complex clinicopathological information (namely, the levels of a panel of miRNA biomarkers) in a biological specimen into a distinct nanopore ‘signature of disease’, offering promising potential to revolutionize the field of clinical diagnostics and ‘liquid biopsy’ tests. To achieve this ultimate goal, a number of objectives are achieved:

### Develop a Versatile and Reliable Platform for Single-Plex Biomarker Information Encoding.

The first objective is to develop a platform which can encode clinically relevant information on a carrier dsDNA molecule. The ability to ‘write’ clinically-relevant information is based upon the design, synthesis and validation of a novel PNA-based 2D hybridization probe with dual-functionality: one motif (the anchor-PNA) enables dsDNA carrier invasion and the other motif (capture-PNA) enables sequence-specific miRNA detection. The ability to flexibly design and engineer dual-function PNA-based probes which can invade dsDNA in a sequence-specific manner and capture target biomarkers gives this platform versatility and novelty.

A key requirement for advanced diagnostic tools is the ability to do multiplexing, i.e. detect multiple validated biomarkers in a single test using a single biological sample. The ability to design and engineer modified PNA probes to invade any dsDNA sequence in a highly selective manner provides flexibility of design and application. This novel approach to achieve location-based encoding of information along a DNA strand enables the platform's powerful and novel multiplexing capability based on the location of the probe along the carrier.

The ability to ‘read’ the encoded information on biomarker levels (such as miRNA) is provides improved sensitivity in the capture and detection of low concentrations of miRNA targets and DNA carriers in the analyte solution.

Exploiting nanopore technology's intrinsic capabilities, applying them and optimizing them for clinical data decoding is novel and provides a transformative route towards the development of ultrasensitive and advanced diagnostic tools.

Among the various liquid biopsy biomarkers, the system can be validated with miRNA due to its promising potential in clinical diagnosis and prognosis of cancer, which has been hindered by the complexity, cost, and limited multiplexing capability of existing gold-standard techniques. The disclosed system addresses these shortcomings in a novel and elegant way is thus to validate the platform's ability to encode information about miRNA expression in a clinically-relevant sample or system (including up-regulated miRNAs, down-regulated miRNA as well as control miRNAs).

The disclosed system is designed to include a sophisticated biomarker data encoding and decoding tool based on a 2D hybridization complex of dsDNA, PNA and miRNA units, with dsDNA acting as the carrier molecule, PNA-PNA conjugate acting as the location-encoded probe, and miRNA as the target biomarker. As presented in FIG. 2, the dsDNA carrier is spatially decorated along its length with multiple bis-hetero-functional polymer-linked anchor-PNA/capture-PNA conjugates at specific locations: the anchor-PNA motif invades the carrier at a specific site (location encoded) while the capture-PNA motif is orthogonally detect the 3′ end of a miRNA target. A dendron-linked PNA amplifier is used to target the 5′ end of the captured miRNA generating a detectable characteristic signal on the nanopore current-time trace. As a result, this technology can translate clinicopathological information about a cancer type (specifically, the dysregulation of a miRNA biomarker panel) into a characteristic signal, and the disclosed system is able to easily decode this information in a quick, high-throughput and simple manner.

PNA sequences and dendron structures are synthesized based on optimized protocols. FIG. 3A-C.

Proof-of-Concept Study to Prepare dsDNA Carrier.

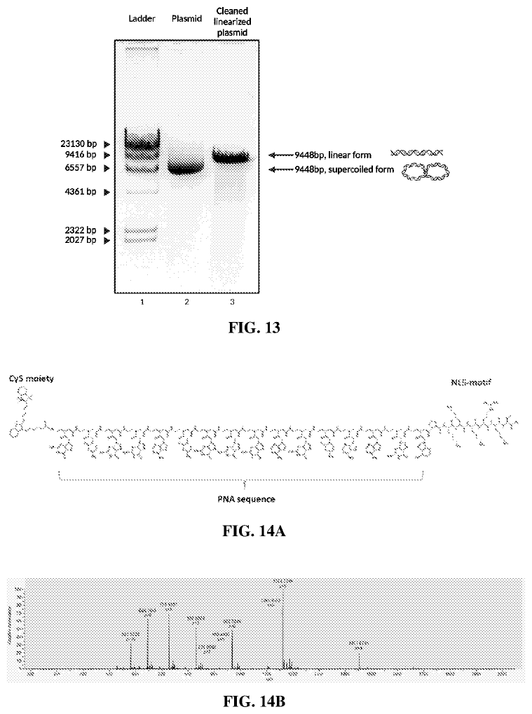

The dsDNA carrier can be prepared according to the method described above in Section II (A) Instructions and Considerations for Designing and Preparing DNA Carriers. FIG. 13 shows the proof-of-concept results which demonstrate the ability to culture the plasmid-containing bacteria, extract, and linearize an exemplary DNA carrier with high yield and purity. From a 60 mL bacterial culture, about 40 μg of carrier DNA was produced which exceeds the requirements for single-molecule methods such as nanopore studies. Thus, this amount is enough in some forms, to conduct at least 25 experiments, each with a statistically significant number of reads.

### Proof-of-Concept Study to Synthesize Anchor-PNA.

### Study 1:

The anchor-PNA probe was synthesized according to the method described above in Section II(B) Instructions and Considerations for Designing and Preparing XNA-based Probes.

FIGS. 14A and 14B demonstrate the successful synthesis of an anchor-PNA probe with an NLS-motif and Cy5 fluorescent tag. The 16-mer sequence was designed to be complementary to a specific site along the prepared carrier DNA. The Cy5-PNA-NLS structure (FIG. 14A) and corresponding LCMS data (FIG. 14B) confirming its successful synthesis are presented, demonstrating that the advanced anchor-PNA probes can be designed and synthesized. The anchor-PNA probes, can serve as a foundation of the “anchor” or ‘hook’ component of the XNA-based or fishing probes.

### Study 2:

A second anchor motif of the probes was designed which can serve to bind the carrier at sequence-specific sites enabling location-based encoding. For this proof-of-concept study, the anchor motif was designed to exploit the invasion capacity of the γ-peptide nucleic acid (γ-PNA) helix, advanced with the invasion-promoting capacity of the short Nuclear Localization Signal (NLS) peptide 4,5. γ-PNA is an advanced class of XNA oligomers that contains a chiral center through modification at the γ-position of the N-(2-aminoethyl) glycine backbone. This modification leads to the preorganization of the γ-PNA into a right-handed helix6 enabling duplex invasion and binding to complementary dsDNA without any sequence limitations. Additionally, the functional peptide, NLS, is a useful conjugation motif that can both thermodynamically and kinetically promote duplex invasion of dsDNA even under high ionic strength conditions.5 NLS is a positively charged peptide rich in basic amino acids like lysine and arginine, enabling it to interact strongly with DNA under neutral pH conditions. In the present design, XNA-based anchor probes, called γ-PNA-NLS, were designed and synthesized, combining the merits of both invasion strategies: the γ-PNA chiral structure and the NLS motif, as validated by LCMS (FIGS. 20A-20D).

In addition to its duplex invasion capacity, the XNA anchor probes have to possess the capacity to bind target biomarkers. This functionality stems from the bioreceptor motif of the probe to capture target biomarkers. As a proof of principle, streptavidin with R-Phycoerythrin Conjugate (SAPE, Thermo Fisher S866) was implemented as a proxy target biomarker, and biotin was chosen as a model bioreceptor (FIG. 20C and FIG. 20D showing chemical structure and LCMS, respectively).7-9 In this instance, the bifunctional probe containing the bioreceptor motif and XNA ‘anchor’ motif, were chemically linked with a short PEG linker.

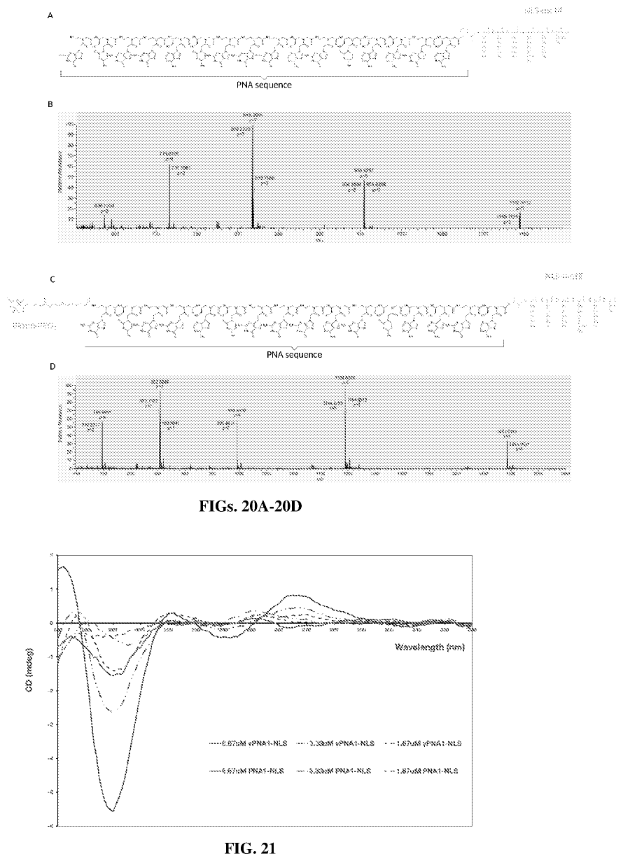

The gamma (γ) modifications were introduced in the PNA backbone as L-serine side chains in the adenine and cytosine residues, whereas the NLS motif was presented at the C-end of the probe sequence. The probe's secondary structure has been confirmed by CD spectroscopy, which reveals a distinct CD signature (FIG. 21), indicating a common folding pattern and secondary helical structure in the γ-PNA and the conventional PNA^{10,11}. Both PNA¹-NLS and γPNA¹-NLS adopted biphasic exciton coupling patterns characteristic of right-handed helices (FIG. 21). However, it was observed that compared to the conventional PNA (solid red trace) the γ-modified PNA (solid blue trace) shows a greater tendency to form a right-handed helical structure which can explain its greater ability for duplex invasion (FIG. 21).

### Validation of the Location-Encoding Capability of the XNA Probes

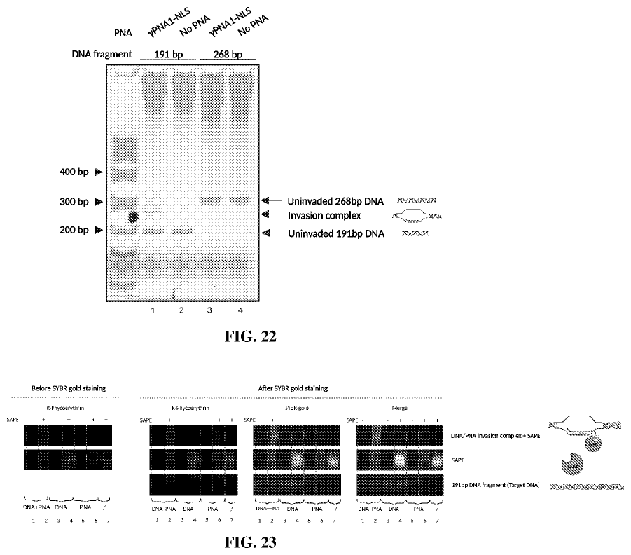

For the successful configuration of the single-molecular sensor, the dsDNA carrier can be spatially decorated along its length with probes in a location-encoded manner. This ability stems from the sequence-specific duplex invasion capacity of the disclosed XNA anchor probes based on γPNA¹-NLS. To prove this ability, gel shift assays were conducted to investigate the electrophoretic gel shift of short fragments of DNA with or without probe binding, wherein a PNA-DNA complex should result in lower electrophoretic mobility compared to an equivalent bare i.e., uninvaded DNA. As a proof-of-concept, two short DNA fragments were isolated from the DNA carrier by double enzyme restriction digestion: (1) a 191 bp DNA fragment containing the target sequence for the anchor motif, and (2) a 268 bp DNA fragment without the target sequence. As presented in FIG. 22, a distinct band appeared in the gel around a size of 300 bp only when the anchor motif was combined with the target 191 bp DNA fragment but not when it was combined with the other 268 bp fragment, indicating that the anchor probe possesses sequence-specific duplex invasion capacity.

### Validating the System's Biomarker Detection Capacity

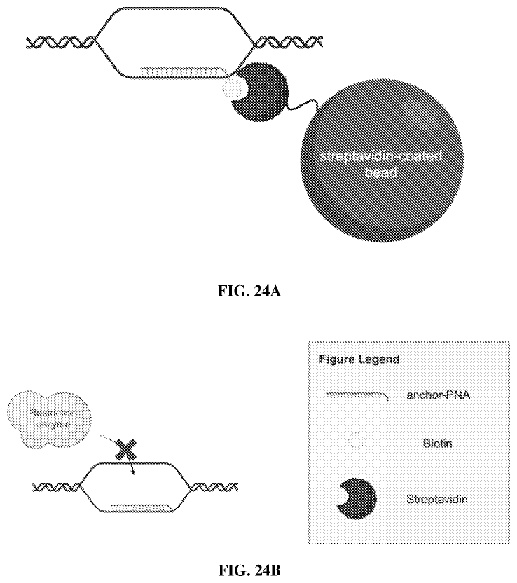

Additional studies were done to validate the bifunctional probe's capability to not only bind the carrier but also capture target biomarkers. SAPE was used as a model biomarker in these studies due to its high fluorescence quantum yield and larger molecular weight, facilitating validation studies via gel electrophoresis and fluorescence imaging. As shown in FIG. 23, when the bifunctional probe (biotin served as bioreceptor motif and γPNA-NLS served as XNA anchor motif) was combined with the carrier DNA and model target biomarker (SAPE), a distinctive band appeared in the polyacrylamide gel. This band was absent in all controls and can thus serves as validation for the successful formation of a multi-component complex composed of the DNA carrier, the bifunctional probe and the model target biomarker. Importantly, the distinctive invasion band shows a fluorescence signal in two fluorescence channels (red and yellow): the yellow channel is associated with the SYBR gold staining of DNA and the red channel (R-PE channel) is indicative of SAPE presence (FIG. 23).

### Investigation of Invasion Conditions: Temperature and Ionic Strength

In order to test the range of possible conditions under which invasion can occur, invasion temperature and ionic strength were tested. The XNA-based probe was designed to have the ability to open the dsDNA helix and form a DNA-XNA-DNA triplex at the invasion site. Here, the 191 bp fragment containing the target sequence to the XNA-based probe was isolated from the ultralong DNA carrier, and the gel shift assay was used to evaluate the invasion ability, as shown in FIGS. 18A-18C. Heating helps break the hydrogen bond inside the dsDNA thus favored by the XNA-based probe invasion. However, if dsDNA is overheated dsDNA fully opened into individual ssDNA, causing 1) XNA-based probe to just simply bind with DNA, not invading into dsDNA (i.e., the difference between invasion and hybridization), leading to “false positive” invasion of tested dsDNA, and 2) the overheated DNA forms complicated high-order structure, which might become noise and affect the nanopore detection.

Another experiment was done to test the stability of DNA/XNA-based probes in salt conditions mimicking nanopore electrolyte conditions. 1M KCl and 0.1M KCl are both commonly used concentrations electrolytes in solid-state nanopore. After performing the invasion reaction at 55 °C for 2 hours, KCl with the indicated concentration was added to the invasion complex and incubated overnight at 4 °C. FIG. 19 shows a clear trend of invaded γPNA¹-NLS remaining invaded in KCl condition at 4 °C overnight under certain ionic concentrations. As shown in FIG. 19, 0.2M is an ideal KCl concentration for γPNA¹-NLS to stay on the invaded DNA, which means that the ionic strength at this point is not enough to damage the invasion and the invaded PNA-dsDNA complex can be stable if the nanopore electrolyte concentration (if KCl is applied) as high as 0.2M. Cy5-PNA¹-NLS with dsDNA invasion complex is unable to stay stable at ionic concentrations approaching 0.1M KCl and above (FIG. 19), with the invasion being at low efficiency when KCl is not present.

### Proof-of-Concept Studies to Synthesize and Validate Five Location-Encoded Anchor-PNAS.

A single anchor-PNA is prepared and its ability to invade the carrier dsDNA (forming a PNA/dsDNA invasion complex) at sequence-specific locations along the carrier is validated. The initial model for the dsDNA carrier can be a short sequence (~300 bp) and is designed to contain a single 22-nt motif with the required sequence for anchor-PNA single invasion. The length of the DNA carrier is based on the following criteria: (1) the DNA carrier must be long enough to allow enough time for single-molecule detection i.e., >2.5 kbp and (2) shorter than about 50 kbp for easier experimental handling. The maximum PNA length is limited by synthesis methods. In some forms, the maximum PNA length is about 40-mer and the minimum PNA length is limited by adequate specificity for binding to the target.

4 more anchor-PNAs with varying inter-anchor-PNA distance are prepared to characterize the longitudinal resolution of single molecule sensing. For this stage, the anchor-PNAs are tagged with a bulky molecule for nanopore analysis and will be fluorescently labeled to facilitate fluorescence-based validation studies only.

Preparation and Validation of a Single Fluorescently-Labeled Anchor-PNA Tagged with a Bulky Moiety (Dendron).

The formation of the single PNA/dsDNA invasion complex is based on PNA binding to its complementary strand of dsDNA through Watson-Crick base pairing. However, owing to thermodynamic constraints (Liang, X, et al., From Fundamentals to Practical Applications. BCSJ 2021, 94, 1737-1756), ordinary PNA structures cannot form stable complexes without chemical modifications (Sugiyama, T., Molecules 2014, 18, 287-310). Typical examples are γ-modification of PNA backbone (Sacui, I., et al., J. Am. Chem. Soc. 2015, 137, 8603-8610., PNA conjugation with positively charged amino acids (Hansen, M. E., et al., Nucleic Acids Res. 2009, 37, 4498-4507), bis-PNA molecules (two PNA oligomers connected by a flexible linker) (Singer, A., et al., Nano Lett. 2010, 10, 738-742), and the use of other pseudo-complementary nucleobases in a double duplex invasion strategy (Lohse, J., et al., Proc. Natl. Acad. Sci. U.S.A 1999, 96, 11804-11808). Unlike other strategies, the γ-PNA strategy is one of the simplest and most promising as there are no restrictions on the target sequence (i.e. mixed purine or pyrimidine sequences can be targeted) (Nölling, J et al., MBio 2016, 7). In addition to their neutral backbone (like standard PNA monomers), which reduces electrostatic repulsions or enthalpic barrier, γ-PNA monomers contain a stereogenic center at the γ-position, which forces the oligomer to pre-organize into a right-handed helix. This preorganization reduces the entropic barrier during duplex formation to a complementary strand.

Considering their superior performance and single-nucleotide specificity owing to their combined enthalpic and entropic advantages, the γ-PNAs is selected for duplex invasion of the dsDNA carrier. Other conventional PNA probes (with/without modifications) may also be used for invasion. As a platform in these studies, a 16-nt PNA with some γ modifications and a peptide motif is used³⁷. The sequence is as follows: K-AAATAAATACTTATAGCAAAAA-K (SEQ ID NO:6) is used as platform in the studies. The second and the third PNA probes are responsible for binding to the specific biomarker of choice. These can be conventional PNA or modified PNA as long as it is complementary to the target biomarker. Alternatively, instead of the two PNA probes highlighted, other bioreceptors can be added to target other biomarkers, e,g., an antibody or antibody mimic (such as a nanobody) can be used to target protein-based biomarkers.

As a novel approach to facilitate single-molecule detection of the dsDNA-PNA complex, the anchor-PNA probe will be tagged with a bulky and rigid molecule (for example, a polylysine dendron (Al-Jamal, K. J. Drug Deliv. Sci. Technol. 2005, 15, 11-18), and it will be fluorescently labeled to facilitate validation studies. To prepare this dendron-linked PNA, the anchor-PNA motif will be extended with a linker using a heterobifunctional polymer of DBCO-PEG-Mal, and then conjugated to the dendron of poly(lysine) with a thiol core and acetylated amine-ends (FIG. 5). To achieve optimal fluorescence for the validation studies two ways for fluorophore tagging on the dendron can be used, such as a single fluorophore incorporated in the dendron core or a number of fluorophores conjugated on the dendron surface using its amine-ends. Synthesis can be validated via HPLC and mass spectrometry, and successful invasion of the anchor-PNA can be studied using the methods of gel electrophoresis (notably gel shift assays) (He, G.; Rapireddy, S, et al., J. Am. Chem. Soc. 2009, 131, 12088-12090), fluorescence imaging, CD spectroscopy and melting curves (Demidov, V. V.; et al., Proc. Natl. Acad. Sci. U.S.A 2002, 99, 5953-5958).

Preparation of a Series of Different Anchor-PNAs, Assigned with Different Fluorescent Dyes, Respectively, for Optimization of the Inter-Anchor-PNA Distance (Based on Nanopore Longitudinal Resolution)

These anchor-PNAs are designed to be complementary to specific locations along a 10 kbp dsDNA carrier. Successful synthesis and purification of the four anchor-PNAs are validated as previously discussed. In this task, the inter-anchor-PNA distance is controlled and varied systematically, starting with a distance of 1000 bp, and optimizing based on the single-molecule sensing method longitudinal resolution (i.e. ability to discriminate between neighboring probes along the dsDNA carrier).

Further experiments explore the detection of the PNA/DNA complexes through single-molecule sensing techniques, their signals, and the longitudinal resolution of the disclosed detection system.

Nanopore detection of PNA-DNA complex: Previous work has demonstrated the detection of a different type of the PNA/DNA complex (namely, bis-PNA/DNA) with nanopores (Singer, A, et al., Nano Lett. 2010, 10, 738-742). A major limitation of using bis-PNA for duplex invasion is that it poses sequence limitations on the binding sites (specifically, the binding site must consist of purines in one strand and pyrimidines in the other). The disclosed novel and versatile design of the anchor-PNA, is preferably based on Y-PNA modifications.

Resolution along the DNA carrier is an important parameter that will inform the design and the development of our essay is the longitudinal resolution along the DNA complex. It is the minimal distance between two anchor-PNAs along the carrier dsDNA that would be detected as two separate ionic-current signals in the nanopore detector. It is expected that the resolution should be at least 2000 base pairs, but more likely 1000 base pairs. If achieving Longer DNA carriers can be used to improve upon resolution.

Studies herein include synthesis and assembly the 2D hybridization complex for a single-plex system (designed to target miR-141) (FIG. 6). This includes the synthesis of the capture-PNA and its covalent linkage to the anchor-PNA (Task 1), followed by synthesis of the amplifier-PNA (Task 2). Task 3 studies the self-assembly of this PNA/PNA conjugate onto the dsDNA carrier followed by the orthogonal capture of the target miRNA, thereby illustrating the novel 2D Hybridization complex for sensing.

Preparation of the polymer-linked anchor-PNA/capture-PNA conjugate is shown in FIG. 7. The anchor PNA can be synthesized with an azido-end functionality, and the capture-PNA can be ended with a cysteine residue, providing a thiol functionality. Subsequently, the two PNA motifs are covalently linked together using the heterobifunctional polymer of α-dibenzocyclooctyne-@-maleimide polyethylene glycol (DBCO-PEG-Mal), through the orthogonal chemical reactions of copper-free Azide-alkyne Huisgen cycloaddition and thiol-maleimide coupling. The linker length can be varied to optimize capture efficiency without affecting invasion efficiency.

### Preparation of the Amplifier-PNA

The dendron-linked amplifier-PNA can be prepared using the same strategy as the preparation of the bulky anchor-PNA in Stage 1. The bulky structure of the dendron and its size can be varied and optimized to facilitate detection with high signal-to-noise ratio and sufficient contrast using the nanopore system. The sequences for the capture-PNA and amplifier-PNA for the single-plex system are chosen and designed to target miR-141. Specifically, the sequence for the target miR-141 is 5′ AACACTGTCTGGTAAAGATGG 3′ (SEQ ID NO:7), while the sequences for the capture-PNA and amplifier-PNAs (C- to N-terminus) are CATTTCT and TGTGACA, respectively. The underlined text in the miRNA sequence indicates the nucleotides where each PNA binds the target miRNA.

Validating the self-assembly of the probe complex involves three steps (i) anchor-PNA invasion of the dsDNA carrier, (ii) capture-PNA detection of the target miRNA, and (iii) hybridization of the amplifier-PNA to the other end of the target miRNA. The 2D hybridization complex of the novel decoding tool is self-assembled using the above discussed components and sequences. Its formation under various physicochemical conditions is studied and validated, thus illustrating the concept for a novel sensing platform based on a 2D hybridization complex.

A crucial performance parameter in the nanopore detection system is its lateral contrast. The nanopore signal depends on the cross-sectional area of the molecule residing with the nanopore at that time—and the lateral contrast determines the minimal difference in geometry along the DNA that leads to distinctive difference in the ionic signal (Scheme 2). The disclosed system is able to detect the difference in the ionic signal between the carrier DNA, the 2D hybridization complex (i.e. PNA/DNA complex) without the target miRNA (negative signal), and the one with the hybridized miRNA target (positive signal). The contrast depends on parameters, such as DNA translocation speed, bandwidth of our electrical detection instruments, signal-to-noise, nanopore geometry (Garaj, S.; et al., Proc. Natl. Acad. Sci. U.S.A 2013, 110, 12192-12196), etc. Optimization strategies to increase the contrast focus on electronics, nanopore chip design and nanopore geometry (size, thickness, material, etc.). Based on previous experiments with DNA complexes (Storm, A. J.; et al., Nat. Mater. 2003, 2, 537), and experiments with DNA knots (Kumar Sharma, R et al. Nat. Commun. 2019, 10, 1-9.), the contrast should be sufficient to detect the difference between miRNA-hybridized and unhybridized 2D hybridization complexes on the carrier DNA. In some forms, bulky amplifier-PNAs to bind the other end of the target miRNA molecules for improved contrast.

A Location-Encoded Multiplex System for miRNA Detection

Herein, a five capture-PNA/amplifier-PNA pairs, designed to target five clinically-relevant miRNAs (Al Sulaiman, D., et al., ACS Sensors 2021), is disclosed (FIG. 8): 1 positive control miRNA (cel-miR-54), 1 negative control miRNA (cel-miR-39), and 3 endogenous miRNAs which are known to be dysregulated in breast cancer patients: miR-21, miR-145 and miR-16 (Zhu, et al., BMS Res. Notes, 2009, 2, 890). In particular, miR-21 is one of the most significantly upregulated miRNAs in breast cancer. It acts as an oncogene and is associated with advanced cancer stage and poor prognosis (Ng, E. K. et al., PLoS One 2013, 8, e53141). Conversely, miR-145 acts as a tumor suppressor and is downregulated in breast cancer patients (Zheng, et al., Tumor Biology, 2016, 37, 8189-8196). Lastly, miR-16 serum expression levels are significantly higher in breast cancer patients compared to healthy controls (Usmani, A., et al., J. Pak. Med. Assoc. 2017, 67, 446-450). The decoding ability of the system is based on studying test samples with different biomarker concentrations and ratios. Specifically, sample solutions with known concentration profiles of the five miRNA targets are prepared. Examples are presented in Table 1, showing the relative ratios of the 5 miRNAs, encoded at different locations along the dsDNA carrier. These model samples can be used to investigate the ability of the nanopore system to recapitulate the biomarker dysregulation pattern as designed.

Table 1. Relative ratios of 5 miRNAs in different test samples. The five miRNAs will be encoded at different locations along the dsDNA carrier, as optimized in Stage 1 based on inter-anchor PNA distance. For example, location 1 will encode the positive control cel-miR-54, whereas location 5 will encode miR-16. As an example, the relative ratios of the five miRNAs can be varied as outlined.

| | Location on dsDNA carrier | | | | |
|---|---|---|---|---|---|
| | 1 | 2 | 3 | 4 | 5 |
| Sample | cel-miR-54 | cel-miR-39 | miR-21 | miR-145 | miR-16 |
| 1 | 1 | 0 | 0 | 0 | 0 |
| 2 | 1 | 1 | 1 | 1 | 1 |
| 3 | 1 | 3 | 0 | 3 | 1 |
| 4 | 0 | 1 | 2 | 5 | 10 |

The nanopore detector system disclosed herein is used to analyze the 5-plex spatially-encoded carrier DNA molecules discussed above. Longer dsDNA carriers can be used (as long as lambda DNA); an increase in the size of the bulky moiety attached to the amplifier-PNA (this end indicates the nanopore signal ‘read’ direction, since the DNA molecule can enter the nanopore from either side,) and thereby increase the volume of the hybridization complex facilitating enhanced nanopore contrast.

The nanopores' system to decode clinicopathological information using model solutions simulating the dysregulation profiles found in the biofluid samples of cancer patients is tested as shown on FIG. 9. For all solutions, the negative control cel-miR-39 will not be added, while the positive control cel-miR-54 are added at a specific detectable concentration (e.g. 300 μM). The levels of the three other miRNA will be varied to model different dysregulation patterns, observed in patient samples. For example, Sample 1 can simulate a cancer patient wherein miR-21 and miR-16 are upregulated while miR-145 is downregulated, compared to a healthy patient sample. Sample 2 can be designed to simulate the miRNA profile of a healthy patient. These solutions are prepared and labeled with a code for successive testing with the nanopore system. The ability of the system to decode clinicopathological information about biomarker dysregulation presents a novel and highly promising approach for simple, sensitive and effective disease diagnosis.

### REFERENCES

(1) Cancer. https://www.who.int/news-room/fact-sheets/detail/cancer (accessed 2023 Sep. 11).
(2) Alqahtani, W. S.; et al., Epidemiology of Cancer in Saudi Arabia Thru 2010-2019: A Systematic Review with Constrained Meta-Analysis. AIMS Public Health 2020, 7 (3), 679-696.
(3) Alessy, S. A.; Al Waheidi, S. Journal of Cancer Policy 2020, 26, 100250.
(4) World Health Organization. Guide to Early Cancer Diagnosis; 2017.
(5) Cancer Survival in England, cancers diagnosed 2016 to 2020, followed up to 2021. NHS Digital. https://digital.nhs.uk/data-and-information/publications/statistical/cancer-survival-in-england/cancers-diagnosed-2016-to-2020-followed-up-to-2021 (accessed 2023 Sep. 11).
(6) Giaquinto, A. N.; et al., Breast Cancer Statistics, 2022. CA Cancer J. Clin. 2022, 72 (6), 524-541.
(7) Connal, S.; et al., Liquid Biopsies: The Future of Cancer Early Detection. J. Transl. Med. 2023, 21 (1), 118.
(8) Karaglani, M.; et al., J. Clin. Med. Res. 2022, 11 (4).
(9) Zhu, W.; Li, X. Methods Mol. Biol. 2023, 2695, 279-293.
(10) Lin, L. H.; et al., Mod. Pathol. 2021, 34 (12), 2168-2174.
(11) Ambros, V. Nature 2004, 431 (7006), 350-355.
(12) Bartel, D. P., Cell 2004, 116 (2), 281-297.
(13) Calin, G. A.; Croce, C. M., Nat. Rev. Cancer 2006, 6 (11), 857-866.
(14) Lu, J.; et al., MicroRNA Expression Profiles Classify Human Cancers. Nature 2005, 435 (7043), 834-838.
(15) Iorio, M. V.; et al, MicroRNA Gene Expression Deregulation in Human Breast Cancer. Cancer Res. 2005, 65 (16), 7065-7070.
(16) Calin, G. A.; et al., A MicroRNA Signature Associated with Prognosis and Progression in Chronic Lymphocytic Leukemia. N. Engl. J. Med. 2005, 353 (17), 1793-1801.
(17) Sur, D.; Advani, S.; Braithwaite, D., Front. Med. 2022, 9, 915226.
(18) Pritchard, C. C.; et al., Nat. Rev. Genet. 2012, 13 (5), 358-369.
(19) Liu, C. G.; et al., Nat. Protoc. 2008, 3 (4), 563-578.
(20) Kulkarni, M. M, Curr. Protoc. Mol. Biol. 2011, Chapter 25, Unit25B.10.
(21) Yuan, Z.; et al., Nanoscale Res. Lett. 2018, 13 (1), 56.
(22) Tang, Z.; et al., Fabrications, Applications and Challenges of Solid-State Nanopores: A Mini Review. Nanomaterials and Nanotechnology 2016, 6, 35.
(23) Branton, D.; et al., Nanometric Material Having A Nanopore Enabling High-Sensitivity Molecular Detection and Analysis, 2020. https://patents.google.com/patent/US20200158712A1/en.
(24) Sharma, R. K.; et al., Nano Lett. 2021, 21 (9), 3772-3779.
(25) Sulaiman, A.; Zaid, D. Imperial College London, 2019. https://doi.org/10.25560/81655.
(26) Bošković, F.; et al., Nat. Nanotechnol. 2023, 18 (3), 290-298.
(27) Chen, K. et al., ACS Nano 2021, 15 (2), 2679-2685.
(28) Bošković, F.; Keyser, U. F. Nat. Chem. 2022, 14 (11), 1258-1264.
(29) Zhu, J.; et al., Nano Lett. 2022, 22 (12), 4993-4998.
(30) Chen, K.; et al., Nano Lett. 2020, 20 (5), 3754-3760.
(31) Morin, T. J.; et al., PLoS One 2016, 11 (5), e0154426.
(32) Singer, A. Nano Lett. 2012, 12 (3), 1722-1728.
(33) Weckman, N. E.; et al., ACS Sens 2019, 4 (8), 2065-2072.
(34) Stanley, P. M.; et al., Biotechnol. Adv. 2020, 45, 107639.
(35) Bornholt, J.; et al., ASPLOS '16; Association for Computing Machinery: New York, NY, USA, 2016; pp 637-649.
(36) Chen, C.; et al., Front. Genet. 2022, 13, 1064715.
(37) Zhang, C.; et al.,
(38) Louie, M.; et al., CMAJ 2000, 163 (3), 301-309.
(39) Silva, F.; et al., J. Microbiol. Biotechnol. 2009, 19 (11), 1408-1414.
(40) Wang, Y.; et al., Nat. Biotechnol. 2021, 39 (11), 1348-1365.
(41) Kono, N, et al., Dev. Growth Differ. 2019, 61 (5), 316-326.
(42) Pal, A.; et al., PLoS Comput. Biol. 2019, 15 (4), e1006768.
(43) Wojciechowska, M.; et al., J. Molecules 2020, 25 (3).
(44) Wang, J. PNA Biosensors for Nucleic Acid Detection. Curr. Issues Mol. Biol. 1999, 1 (1-2), 117-122.
(45) Shakeel, et al., J. Chem. Technol. Biotechnol. 2006, 81 (6), 892-899.
(46) Uhlmann, et al., Angew. Chem. Int. Ed Engl. 1998, 37 (20), 2796-2823.
(47) Nielsen, P. E.; et al., Curr. Issues Mol. Biol. 1999, 1 (1-2), 89-104.
(48) Liang, X.; et al., BCSJ 2021, 94 (6), 1737-1756.
(49) Takahashi, S.; et al., Acc. Chem. Res. 2021, 54 (9), 2110-2120.
(50) Kumar Sharma, R.; et al., Nat. Commun. 2019, 10 (1), 1-9.
(51) Al Sulaiman, D.; et al., ACS Nano 2019, 13 (8), 9620-9628.
(52) Kim, E.; Koo, H. Chem. Sci. 2019, 10 (34), 7835-7851.
(53) Northrop, B. H.; et al., Polym. Chem. 2015, 6 (18), 3415-3430.
(54) QIAGEN. QIAGEN Plasmid Purification Handbook. https://www.qiagen.com/us/resources/resourcedetail?id=0bd0c5fb-c271-43e7-af43-32d539 374fa9&lang=en (accessed 2023 Sep. 17).
(55) Rapireddy, S.; et al., J.Am. Chem. Soc. 2007, 129 (50), 15596-15600.
(56) Zhang, X.; et al., Nucleic Acids Res. 2000, 28 (17), 3332-3338.
(57) Lu, J.; et al., Cell Commun. Signal. 2021, 19 (1), 60.
(58) Branden, L. J.; et al., Nat. Biotechnol. 1999, 17 (8), 784-787.
(59) Aiba, Y.; et al., Org. Biomol. Chem. 2013, 11 (32), 5233-5238.
(60) Al-Jamal, K. T.; et al., J. Drug Deliv. Sci. Technol. 2005, 15 (1), 11-18.
(61) Dennler, P.; et al., Antibodies 2015, 4 (3), 197-224.
(62) Da Pieve, C.; et al., Bioconjug. Chem. 2010, 21 (1), 169-174.
(63) Al Sulaiman, Angew. Chem. Int. Ed Engl. 2017, 56 (19), 5247-5251.
(64) Garaj, Proc. Natl. Acad. Sci. U.S.A 2013, 110 (30), 12192-12196.
(65) Li, et al., Tumour Biol. 2016, 37 (7), 8765-8773.
(66) Liu, T.; et al. Ann. Med. 2021, 53 (1), 87-102.
(67) Hao, J.-P., Eur. Rev. Med. Pharmacol. Sci. 2018, 22 (24), 8649-8656.
(68) Gorska, et al., Angew. Chem. Int. Ed Engl. 2013, 52 (27), 6820-6843.
(69) Bonifazi, Artif. DNA PNA XNA 2012, 3 (3), 112-122.

1. A composition specific for the multiplex detection of target biomarkers of interest, comprising:
i) one or more bifunctional Xeno Nucleic acid (XNA)-based probes wherein each probe comprises a first domain (an anchor) and a second domain (a hook),
wherein anchor comprises a XNA and the hook comprises a binding partner to an analyte of interest,
wherein the anchor and the hook are optionally separated by a linker; and
(ii) a double stranded DNA carrier molecule comprising sequences complementary to the one or more anchors (anchor-complimentary sequences).

2. The composition of claim 1, wherein the linker is: (a) a flexible linker; (b) a polymeric linker, wherein the polymeric linker comprises polyethylene glycol (PEG), optionally, wherein the polymeric linker comprises two PEG units; or (c) a peptide linker.

3. (canceled)

4. (canceled)

5. The composition of claim 2, wherein the linker is selected from the group consisting of SEQ ID NO: 8-SEQ ID NO: 16.

6. The composition of claim 1, wherein the binding partner is a synthetic molecule or a natural molecule.

7. The composition of claim 6, wherein the binding partner is a nucleic acid binding partner, peptide binding partner, protein binding partner, or a carbohydrate binding partner.

8. The composition of claim 7, wherein the nucleic acid binding partner is a peptide-nucleic acid or a nucleic acid aptamer.

9. The composition of claim 7, wherein the peptide or protein binding partner is an antibody, peptide aptamer, or nanobody.

10. The composition of claim 1, wherein the anchor comprises a peptide nucleic acid (PNA).

11. The composition of claim 1, wherein the DNA carrier has a size ranging from 2.5 kbp to 50 kbp.

12. The composition of claim 1, wherein the one or sequence sequences complementary to sequences in the anchor are separated by at least 400-1000 bp.

13. The composition of claim 1, wherein the DNA carrier has along its length, at least five anchor-complimentary sequences, wherein the five anchor-complimentary sequences hybridize to five different anchors.

14. The composition of claim 1, wherein the anchor has a size ranging from 8-mer to 25-mer.

15. The composition of claim 1, wherein the composition further comprises a third component comprising a nucleic acid functionalized with a label, wherein the third component is complementary to a portion of the hook, when the hook is a nucleic acid.

16. The composition of claim 10, wherein the PNA is a γPNA.

17. The composition of claim 1, wherein the anchor comprises K-AAATAAATACTTATAGCAAAAA-K (SEQ ID NO:6).

18. The composition of claim 1, wherein the PNA further comprises a nuclear localization signal.

19. A method for detecting an analyte of interest in a sample, comprising contacting the sample with a composition of claim 1.

20. The method of claim 19, wherein: (a) the sample is a clinical sample, optionally, wherein the sample is a blood sample, plasma sample, tissue sample, and other bodily fluids or (b) an environmental sample.

21. (canceled)

22. The method of claim 19, wherein the analyte is miRNA.

23. The method of claim 19, further comprising subjecting the sample to a nanopore sensor or nanochannel assay.
